(19) United States
(12) Patent Application Publication
(10) Pub. No.: US 2010/0062981 A1
(43) Pub. Date: Mar. 11, 2010

(54) USE OF FIBRINOGEN AS A PROPHYLACTIC TREATMENT TO PREVENT BLEEDING DURING AND AFTER SURGERY AND AS A BIOMARKER TO IDENTIFY PATIENT WITH AN INCREASED RISK FOR EXCESSIVE BLEEDING AND BLOOD TRANSFUSION

(76) Inventors: Anders Jeppsson, Kullavik (SE); Stanko Skrtic, Goteborg (SE); Lennart Bruce, Viken (SE); Jörgen Johnsson, Helsingborg (SE); Thomas Hedner, Gallstad (SE)

Correspondence Address: STOEL RIVES LLP - SLC, 201 SOUTH MAIN STREET, SUITE 1100, ONE UTAH CENTER, SALT LAKE CITY, UT, 84111, US

(21) Appl. No.: 12/525,516

(22) PCT Filed: Jan. 30, 2008
(86) PCT No.: PCT/EP08/00710

§ 371 Date: Nov. 13, 2009

Related U.S. Application Data
(60) Provisional application No. 60/887,441, filed on Jan. 31, 2007.

(30) Foreign Application Priority Data
Aug. 29, 2007 (DK) PA 2007 01232

(51) Int. Cl. A61K 38/36; A61P 7/00; C12Q 1/56

(52) U.S. Cl. 514/12; 435/13

(57) ABSTRACT

The present invention provides a method for preventing peri- and postoperative bleeding in subjects undergoing surgery, in particular subjects with a preoperative fibrinogen plasma level equal to or above the normal range. The method comprises administration of a substance with fibrinogen-like activity to the subject in an amount that result in a circulating fibrinogen plasma level of from about 1.0 g/L. The present invention also provides a method for determining the risk of subjects with a preoperative fibrinogen plasma level equal to or above the normal range to bleed postoperatively. Furthermore, the present invention provides means for predicting the necessity of blood or plasma transfusion after a subject with a preoperative fibrinogen plasma level equal to or above the normal range has been subject to a surgical procedure. The means involves measurement of the fibrinogen level of the subject before surgery and comparing the level with a risk curve.

### FIELD OF THE INVENTION

The present invention provides a method for preventing peri- and/or postoperative bleeding in subjects undergoing a surgical procedure, especially in those situations where blood loss may cause unfavorable systemic and/or local complications for the subject. The method involves administration of a predetermined amount of a substance with fibrinogen-like activity, notably fibrinogen, to adjust the fibrinogen level of the patient typically having a preoperative fibrinogen plasma level equal to or above the lower normal limit, notably within the normal range or slightly above.

The present invention also provides a method for determining the risk of subjects with a preoperative fibrinogen plasma level equal to or above the normal range to bleed postoperatively. The method can also preferentially involve measurement of the plasma fibrinogen level of the subject before the surgical procedure is carried out and comparing the value obtained from a risk estimation or with a risk curve.

Furthermore, the present invention provides means for predicting the necessity of blood or plasma transfusion after a subject with a preoperative fibrinogen plasma level equal to or above the normal range has been subject to a surgical procedure. The means involves measurement of the fibrinogen level of the subject before surgery and comparing the level with a risk curve.

### BACKGROUND OF THE INVENTION

### Perioperative Bleeding

Bleeding remains an important complication of certain complex surgical procedures particularly cardiac operations associated with long bypass times and profound hypothermia. Assessment of the patient preoperatively will identify drug-induced, acquired, or inherited coagulation defects that may contribute to further complicate this problem. Surgery affects coagulation, fibrinolysis as well as platelet function. In patients undergoing cardiac surgery, operative revision procedures due to bleeding, has to be performed in 2% to 6% of patients and is associated with a marked deterioration in general outcome and prognosis (Hartmann et al. Effects of cardiac surgery on hemostasis. Transfus Med Rev. 2006; 20:230-41.

Overall, mortality rates of 0.1% are today observed for surgical procedures, but rates may be as high as 5% to 8% for elective cardiovascular surgery, and even as high as 20% in the presence of severe perioperative or postoperative bleeding (Marietta et al. Pathophysiology of bleeding in surgery. Transplant Proc. 2006; 38:812-4). A number of factors contribute to the acquired hemostatic abnormalities in major surgery such as cardiac surgery, e.g., the use of anticoagulants as well as the activation and consumption of coagulation factors and platelets induced by the extracorporeal circulation (Smith et al Management of bleeding complications in redo cardiac operations. Ann Thorac Surg. 1998; 65:S2-8. Currently, the management of perioperative hemostatic abnormalities is mainly based on the administration of blood components (fresh frozen plasma and platelet concentrates).

Current research indicates that a substantial part of intraoperative and early postoperative bleeding is due to technical factors. This indicates that preoperative work up in order to detect either acquired or congenital coagulopathies will not solve all causes of excess surgical hemorrhage. The principal causes of non-surgical hemostatic perioperative bleeding are a pre-existing undetected bleeding disorder, related to the nature of the operation itself or from coagulation abnormalities arising from massive blood loss. Very often, it is a combination and coexistence of various pathologies. Identifying patients at risk remains a major issue in preventing excessive blood loss.

Uncontrolled bleeding will lead to a combination of hemodilution, hypothermia, consumption of clotting factors, and acidosis, which in turn worsen the clotting process, and further exacerbates the problem in a vicious circle. At present, the standard treatment for surgical bleeding is the rapid control of the source of bleeding by either surgical or radiological techniques (Marietta et al. Pathophysiology of bleeding in surgery. Transplant Proc. 2006; 38:812-4. Blood-derived products as well as hemostatic agents, such as aprotinin and tranexamic acid, may be used to improve hemostatic balance in bleeding patients. Recombinant activated factor VII (rFVIIa) has been reported to be effective for the treatment of surgical or traumatic massive bleeding unresponsive to conventional therapy.

Treatment with human fibrinogen concentrate has been reported in patients with congenital fibrinogen deficiencies such as afibrinogenaemia, heamostatically relevant hypofibrinogenaemia, or dysfibrinogenaemia (Kreuz et al. Efficacy and tolerability of a pasteurised human fibrinogen concentrate in patients with congenital fibrinogen deficiency. Transf Apher Sci 2005; 32:247-53) both to stop ongoing bleedings, as prophylaxis before surgery, or for routine prophylaxis to prevent spontaneous bleeding. Furthermore, in the management of spontaneous bleedings as well as in management of surgery, administration of fibrinogen is advised to patients with hereditary or acquired fibrinogen deficiency according to some guidelines (Bolton-Maggs et al. The rare coagulation disorders—review with guidelines for management from the United Kingdom Haemophilia Centre Doctors' Organisation. Haemophilia 2004; 10:593-628).

A few documents exist, reporting a benefit of administering both FVII and fibrinogen in the treatment of major surgical bleedings in a patient population (U.S. Pat. No. 825,323 and WO 02/055102). Herein, the fibrinogen is administered to act as a substrate for FVII because it is found that administering FVII alone will in some instances be insufficient to produce a clinical significant result. Thus, to provide additionally material for FVII for the clot formation, fibrinogen was co-administered.

However, to the best of the inventors knowledge no one have, until now, administered fibrinogen as a single treatment to patients with a plasma fibrinogen level equal to or above the lower normal limit to prevent perioperative and/or postoperative bleedings. Safety considerations have excluded the administration of fibrinogen to patients with a normal plasma fibrinogen level due to the risk of unwanted thrombi formations within the blood vessels. Bleedings in patients with plasma concentrations within the normal range usually indicates a surgical rather than a coagulopathic cause.

Currently fibrinogen is only available as a plasma-derived product and its availability is thus limited. Hence is it mainly used to treat afibrinogenic conditions.

### DETAILED DESCRIPTION OF THE INVENTION

The present invention addresses the above-mention problem and provides a method for preventing perk and postoperative bleeding in subjects undergoing surgery, in particular subjects with a preoperative fibrinogen plasma level equal to or above the normal range as defined below. The method comprises administration of a substance with fibrinogen-like activity to the subject in an amount that result in a circulating fibrinogen plasma level of from about 1.0 g/L.

As will be apparent from the below description, and from the examples herein, clinical studies are reported, which shows that there is a significant correlation between fibrinogen level and bleeding in subjects with a fibrinogen level within the normal range (2.0 g/L-4.5 g/L). However, it is also reported, e.g. in Example 2, that even in subjects with higher fibrinogen values than the normal range (between 2.4-8.1 g/L in the given example), fibrinogen is a limiting factor. Thus, the present invention does not only relate to a method for preventing perk and postoperative bleeding in subjects with a preoperative fibrinogen plasma level in normal range (as defined below), but also to subjects having a preoperative fibrinogen plasma level above the normal level (2.0 g/l to about 4.5 g/l) such as, e.g. above 2.0 g/L, above 2.5 g/l, above 3.0 g/l, above 3.5 g/l, above 4.0 g/l, above 4.5 g/L, and even above the upper limit of the normal range such as above 5 g/L, such as above 6 g/L, such as above 7 g/L, such as above 8 g/L, such as above 9 g/L, or higher.

In the present context the term “prevention” or “preventing” in connection with “perk and postoperative bleeding” is intended to denote a substantial reduction of perk and postoperative bleeding, i.e. a reduction that is at least 15%, but may be up to 25% or more compared with a control or reference group, which consists of a group that has received no fibrinogen treatment.

The term “perioperative bleeding” is intended to include any bleedings occurring in the time period surrounding a patient's surgical or operative procedure. Before the surgery or operative procedure starts the perioperative bleeding may include bleedings associated with e.g. epidural anaesthesia or other invasive procedures. Depending on the exact circumstances, this may include a time period before the surgery/operation such as e.g. 12 hours before, 5 hours before, 1 hour before or 30 minutes before surgery, but also includes intraoperative (occurring during the surgery/operation) and postoperative bleedings (occurring after surgery).

The term “postoperative bleedings” is intended to mean any bleedings after surgery or operation. In the present context it is intended to cover the time period up to 48 hours after surgery, such as e.g. 12 hours after surgery, 6 hours after surgery, 3 hours after surgery, 1 hour after surgery, or less.

By the term “surgical” or “operative procedure”, used interchangeably herein, is meant to include any invasive procedure in a subject for diagnose or treatment purposes. A surgical procedure may involve the incisions with instruments to repair or arrest disorders, such as diseases, injuries or deformities, in the living body, or the removal or replacement of an organ or tissue. A surgical procedure for diagnostic purposes may for example include invasive imagining techniques and minimally invasive diagnosis techniques comprising endoscopic- and catheter-based interventions.

In the present context, the term “a normal fibrinogen plasma level” means a fibrinogen plasma level in the range of 2.0 to 4.5 g/L in case of a human subject.

In the present context, the term “prior to operation”, as used in e.g. in the claims herein, is intended to exclude patients who receives treatment with fibrinogen substitutions or replacement therapy due to illnesses, in order to adjust the plasma level to the normal level prior the operation. These illnesses may include congenital or acquired fibrinogen deficiencies, such as, but not limited to, afibrinogenaemia, hypofibrinogenaemia, and dysfibrinogenaemia. It is envisaged that the period prior operation may be of at least 1 week, such e.g. 10 days, 2 weeks, 2½ week or more.

Nevertheless, it should also be envisaged, that the prevention treatment according to the invention could be beneficial for subjects with a preoperative unrecognised and untreated congenital or acquired fibrinogen deficiency lying in the lower end of the normal fibrinogen plasma level or minus 20-50% outside the normal level. However, a slight dose adjustment will be required compared to what is described herein, in order to adjust the subjects to a normal fibrinogen plasma level.

In specific embodiments, administration of the amount of the substance with fibrinogen-like activity results in a circulating fibrinogen plasma level of from about 2 g/L to about 10 g/L, about 2 g/L to about 9 g/L, notably from about 2 g/L to about 8 g/L, from about 2.5 g/L to about 7.5 g/L, from about 3 g/L to about 7 g/L, from about 3 g/L to about 6 g/L or from about 3.5 g/L to about 5.5 g/L. In the presently most interesting or preferred embodiments, the resulting fibrinogen plasma level is 5±1 g/L. The fibrinogen level after administration of the substance with fibrinogen-like activity is typically measured in a time period of up to 48 hours after surgery such as in a time period of from 1-24 or 1-12 hours after surgery.

Fibrinogen therapy is used clinically to treat or prevent massive bleeding in patients with inherited or acquired fibrinogen deficiency. However, to the best of the inventors' knowledge fibrinogen has never been used for preventing perk and postoperative bleeding in subjects with a preoperative fibrinogen plasma level in normal range or above. Moreover, to the best of the inventor's knowledge the preoperative fibrinogen level has not been used as an indicator for the need of fibrinogen in order to prevent perk and postoperative bleeding and neither has this level been used as an indicator of a patient's risk of bleeding or its need for blood or plasma transfusion during or after surgery.

In the examples herein clinical studies are reported showing a significant correlation between fibrinogen level and bleeding in patients undergoing cardiac surgery, despite the fact that all patients had preoperative fibrinogen levels within the normal range (2.0 g/L-4.5 g/L), or above (see Examples 1 and 2). The data from Example 2 has enabled the inventors to produce a risk curve for male and female, respectively, i.e. a curve showing the risk for blood transfusion depending on the plasma fibrinogen level before surgery. Moreover, initial clinical studies by the present inventors (see Example 3) have shown that adjustment of the plasma fibrinogen level to at least the upper level of the normal range significantly prevents bleeding after cardiac surgery.

The present invention is primarily based on the results of the clinical studies reported in the Examples herein. However, it is contemplated that the observation also is valid for other types of surgical procedures. Accordingly, the present invention is not limited to the prevention of bleeding after cardiovascular surgery but may be extended to any surgery with a risk of excessive bleeding. Without limiting the invention thereto, such surgery includes any surgery with substantial bleeding risk, cardiovascular surgery, gynaecological surgery, urological surgery, orthopaedic surgery including hip replacement surgery and back surgery; gastrointestinal surgery, transplantations and tumour surgery. However it is also envisaged that a surgery where limited bleeding may cause harm, such as ophthalmic surgery or neurosurgery, will benefit of a treatment according to the invention.

As will be apparent from the given examples herein, the cardiovascular surgery in specific embodiments includes all types of open heart surgery with or without the use of cardiopulmonary bypass (CPB), including e.g coronary artery bypass surgery (CABG), off-pump coronary artery bypass surgery (OPCAB), minimally invasive direct coronary artery bypass surgery (MIDCAB), valve surgery, and aortic surgery or any combinations of the methods mentioned. Furthermore, the surgical procedure also includes cardiovascular surgery or any other surgical intervention including catheter based interventions such as percutaneous coronary intervention (PCI), and percutaneous valve replacements, minimally invasive intervention or any modifications.

In the present context, the term “subject” is intended to include a living organism including a mammal, notably a human.

Moreover, commercially available fibrinogen preparations have been used in the Examples, but it is contemplated that substances having substantially similar activity can be employed. Accordingly, in the present context, the term “a substance with fibrinogen-like activity” is intended to include human or recombinant fibrinogen, a fragment of human or recombinant fibrinogen, a chemically modified fibrinogen or a genetically modified fibrinogen, provided that the derivative of human or recombinant fibrinogen, the chemically modified fibrinogen or the genetically modified fibrinogen has at least 50% of the activity of human fibrinogen. In specific embodiment the substance with fibrinogen-like activity has at least 70% such as at least 75%, at least 80%, at least 85%, at least 90%, at least 95% or at least 99% of the activity of human fibrinogen.

Fibrinogen (alternate names: factor 1, plasma fibrinogen, serum fibrinogen) plays a vital role in a number of physiopathological processes in the body, including inflammation, atherogenesis and thrombogenesis. Fibrinogen is a soluble glycoprotein found in the plasma, with a molecular weight of 340 kDa. It comprises three pairs of non-identical polypeptide chains (alpha, beta and gamma chains) linked to each other by disulphide bonds. Fibrinogen has a biological half-life of about 100 hours and is synthesized predominantly in the liver. Normally, fibrinogen circulates in the plasma at a concentration of approximately 2.0-4.5 g/L.

Plasma fibrinogen is an important component of the coagulation cascade. Fibrinogen is the substrate for fibrin clot formation, which is a template for both thrombin binding and the fibrinolytic system. Fibrinogen binds to platelets to support platelet aggregation and has also a role in wound healing. Virtually all anti-coagulation and anti-platelet drugs used in cardiovascular interventions directly or indirectly affect fibrinogen, its polymerization mechanism, or its target receptors.

Increasing evidence from epidemiological studies suggest that elevated plasma fibrinogen levels are associated with an increased risk of cardiovascular disorders, including ischemic heart disease and stroke. The mechanisms by which fibrinogen may promote atherosclerosis and thrombosis are still not fully understood but fibrinogen affects hemostasis, blood rheology, platelet aggregation, and endothelial function, which all may influence the development of cardiovascular diseases.

As mentioned above, fibrinogen is a glycoprotein. In a specific embodiment of the invention, the substance with fibrinogen-like activity has 75% or more such as 80% or more, 85% or more, 90% or more, 95% or more or 99% or more amino acid identity with human fibrinogen (see e.g. for protein sequences Swiss-Prot/TrEMBL:

http://www.expasy.ch/sprot/sprot-search.html and for amino acid sequences:
http://www.expasy.org/cgi-bin/get-sprot-raw-list.pl?AC=Q32Q65|Q3KPF2|Q4QQH7|Q53Y18|Q9UE34&format=1
<http://www.expasy.org/cgi-bin/get-sprot-raw-list.pl?AC=Q32Q65|Q3KPF2|Q4QQH7|Q53Y18|Q9UE34&format=1>).

In the present context the term “sequence identity of at least about 80%” is intended to indicate that the amino acid sequence of the peptide on average may include up to 2 amino acid alterations per each 10 amino acid residues of the specific amino acid sequence. In other words, to obtain a peptide having an amino acid sequence of at least 80% identity to a specific sequence, up to 20% of the amino acid residues in the subject sequence may be inserted, deleted, or substituted with other amino acid residues.

In addition to the insertion, deletion or substitution of amino acid residues mentioned above, the fibrinogen may comprise other substitutions such as, e.g., “conservative amino acid substitutions”, i.e. substitutions performed within groups of amino acids with similar characteristics, e.g., small amino acids, acidic amino acids, polar amino acids, basic amino acids, hydrophobic amino acids and aromatic amino acids.

A very important and surprising finding of the present invention is that even if the subject undergoing surgery has a preoperative fibrinogen level within the range that is defined as the normal range (i.e. 2.0-4.5 g/L), administration of fibrinogen before surgery has a limiting effect on perk and postoperative bleeding. The target value of the plasma fibrinogen level is contemplated to be in the upper third of the normal range (e.g. 3.5-4-5 g/L) and may even be above the upper limit of the normal range (e.g. up to 10 g/L such as up to 8 g/l, up to 7 g/L, up to 6 g/L or up to or about 5 g/L). At present, the target value is believed to be approximately about 5 g/L, but for humans even a minor increase in plasma fibrinogen level before surgery may reduce the risk of perk and postoperatively bleeding. Given that elevated plasma fibrinogen levels above the normal range is a risk factor for cardiovascular disease, the risk of bleeding must be weighted against the risk for thromboembolic episodes possibly caused by fibrinogen. The half-life of fibrinogen is approximately 3 to 4 days and the substantial outcome risk present for patients who experience massive bleeding or the risk of occurrence of massive bleeding after surgery argue in favour of fibrinogen as a prophylactic measure.

When carrying out the method of the present invention, the subject or patient typically has a plasma fibrinogen level of at the most about 6 g/L before any administration of the substance with fibrinogen-like activity, and—as mentioned above, the subject may have and often has a plasma fibrinogen level within the normal range before any administration of the substance with fibrinogen-like activity.

As demonstrated in the present Examples, a dose of 2 gram of fibrinogen results in a mean increase in plasma fibrinogen of about 0.4-0.75 g/L. More specifically, the plasma fibrinogen level after administration is generally increased with from about 0.1 to about 0.4 g/L such as from about 0.15 to about 0.3 g/L per 1 gram of fibrinogen administered.

This information together with the risk curve shown in FIG. 3 gives a person skilled in the art some guidance in determining an individual dose of the substance with fibrinogen-like activity for an individual patient. Care must of course be taken as the data is based on endogeneous fibrinogen levels and a corresponding curve has not yet been produced in case of exogeneous fibrinogen is administered. However, it is contemplated that the curve can be used as an initial guidance and follow-up studies are on-going.

In accordance with the above, in a method of the invention the substance with fibrinogen-like activity is normally administered in a dose equivalent of from about 0.5 g to about 20 g such as from about 0.5 g to about 15 g, from about 1 g to about 10 g, from about 1 g to about 5 g or from about 1 g to about 2 g of human fibrinogen. If human fibrinogen is not used, then the substance with fibrinogen-like activity can be tested for fibrinogen activity e.g. by testing of how well thrombin cleaves, i.e. generates fibrin, or testing affinity to fibrinogen receptor, and adjust the dose accordingly. In humans fibrinogen has a half-life of 3-4 days and, accordingly, a person skilled in the art will know how to take the degradation/elimination of fibrinogen within the body into consideration when deciding the actual dose and taken into consideration how long in advance of the surgical procedure the fibrinogen is to be administered. Accordingly, fibrinogen can be administered just before surgery or it may be administered several hours or days before. The fibrinogen may also be administered after the surgical procedure in case bleedings occurs, or if there is a risk of bleeding. A person skilled in the art will know how to adjust the dose dependent on the target fibrinogen plasma level at the time the surgery takes place.

In a specific embodiment of the present invention a dose regimen is provided in accordance with the following:

A method according to any one of the preceding claims, wherein the substance with fibrinogen-like activity is administered in accordance with the following:
i) if the plasma fibrinogen level of the subject is in a range of from about 2 g/L-3 g/L then a dose from about 0.30 g/kg body weight to about 0.025 g/kg body weight is administered,
ii) if the plasma fibrinogen level of the subject is in a range of from about 3 g/L-4.5 g/L then a dose from about 0.20 g/kg body weight to about 0.005 g/kg body weight is administered,
iii) if the plasma fibrinogen level of the subject is in a range of from about 4.5 g/L-7 g/L then a dose from about 0.20 g/kg body weight to about 0.005 g/kg body weight is administered.

In a further specific embodiment of the present invention a dose regimen is provided in accordance with the following:
i) if the plasma fibrinogen level of the subject is in a range of from about 2 g/L-3 g/L then a dose from about 0.20 g/kg body weight to about 0.05 g/kg body weight is administered,
ii) if the plasma fibrinogen level of the subject is in a range of from about 3 g/L-4.5 g/L then a dose from about 0.10 g/kg body weight to about 0.02 g/kg body weight is administered,
iii) if the plasma fibrinogen level of the subject is in a range of from about 4.5 g/L-7 g/L then a dose from about 0.10 g/kg body weight to about 0.01 g/kg body weight is administered.

In a subject with a plasma fibrinogen level below the normal level a dose regime is provided in accordance with the following:
i) if the plasma fibrinogen level of the subject is in a range of from about 0.1 g/L-1 g/L then a dose from about 0.40 g/kg body weight to about 0.75 g/kg body weight is administered,
ii) if the plasma fibrinogen level of the subject is in a range of from about 1 g/L-2 g/L then a dose from about 0.35 g/kg body weight to about 0.05 g/kg body weight is administered.

In another embodiment, a dose regime for a subject with a plasma fibrinogen level below the normal level is provided in accordance with the following:
i) if the plasma fibrinogen level of the subject is in a range of from about 0.1 g/L-1 g/L then a dose from about 0.30 g/kg body weight to about 0.15 g/kg body weight is administered,
ii) if the plasma fibrinogen level of the subject is in a range of from about 1 g/L-2 g/L then a dose from about 0.25 g/kg body weight to about 0.10 g/kg body weight is administered.

Due to the long half-life of fibrinogen the above-described dose regimes are applicable for administrations about 24 hours before the surgical procedure or the same day. As described before herein, a person skilled in the art will know how to modify the dose regime if the administration is performed more than 24 hours before the surgical procedure. The dose regimes are also applicable for bleedings occurring after the surgical procedure.

In order to determine whether a patient should be supplemented with fibrinogen before a surgical procedure is carried out, the plasma level is measured before surgery. The point in time when the measurement of plasma fibrinogen level should be made depends inter alia on the condition of the patient and her need for medication. Thus, if the patient needs supply of liquids e.g. by infusion, such a supply will normally influences the plasma fibrinogen level and, accordingly, the plasma fibrinogen level should be measured as late as possible before the surgical procedure is carried out. On the contrary, if the patients are without any need of extra liquid supply, the plasma fibrinogen level is generally very constant and can be measured well before the surgical procedure is carried out. In general, it is contemplated that the plasma fibrinogen level of the subject is measured at the most 2 weeks before a surgical procedure is carried out in order to have reliable data. However, as explained above, there may be situations where the plasma fibrinogen level of the subject is measured at the most 1 week such as, e.g. at the most 6 days, at the most 5 days, at the most 4 days, at the most 3 days, at the most 2 days, at the most 1 day, at the most 18 hours, at the most 12 hours, at the most 6 hours, at the most 3 hours, at the most 2 hours, at the most 1 hour, or at the most 30 minutes before a surgical procedure is carried out.

In order to ensure a suitable fibrinogen plasma level during a surgical procedure and/or in order to minimize the risk of cardiovascular side-effect, the substance with fibrinogen-like activity is normally administered at the most 24 hours before the surgical procedure but it could also be considered to be administered notably earlier taken into account fibrinogens half-life of 3 to 4 days. It is thus envisaged that fibrinogen can be administered as early as at the most 240 hours before the surgical procedure provided that the dose is adjusted taken the biological half-life into consideration and the safety aspects as well. Fibrinogen is typically administered intraveneously, i.e. the plasma concentration is immediately influenced by the amount administered. As explained above, there may be situations where the patient intraveneously is supplemented with liquids and then the plasma fibrinogen level changes dependent on the volume of liquids infused. Accordingly, in many situations it is of importance that the fibrinogen is administered later such as e.g. at the most 18 hours such as, e.g., at the most 12 hours, at the most 9 hours, at the most 6 hours, at the most 3 hours, at the most 1.5 hours or just before or during the surgical procedure.

In a method according to the present invention the subject may receive one or more further administrations of the substance with fibrinogen-like activity e.g. during the surgical procedure or after the surgical procedure, notably up to 2 days after the surgical procedure, in order to reduce or prevent excessive bleeding. Accordingly, such a one or more further administration of the substance with fibrinogen-like activity may take place during the surgical procedure and/or up to 1 day such as, e.g., up to 18 hours, up to 12 hours, up to 6 hours, up to 3 hours, up to 1.5 hours or up to 1 hour after the surgical procedure.

Moreover, there may be situations where the patient will benefit from co-administration (either at the same time or before or after administration of the substance with fibrinogen-like activity) with one or more therapeutically and/or prophylactically active substances. Accordingly, in a further embodiment the method of the present invention comprises administration of a further therapeutically or prophylactically active substance.

The further active substance may be a haemostatic agent such as aprotinin, tranexamic acid, vasopressin or Novoseven. This is relevant when the risk for bleeding is overwhelming for example in patients with inherited bleeding disorders, and it is not expected to be sufficient using a single drug.

The further active substance may also be a platelet inhibitor such as clopidogrel, acetylsalicylic acid, vasopressin, heparin, low molecular weight heparin, warfarin, GP IIb/IIIa modulators, thrombin inhibitor, fibrinogen inducing substance including IL2b. This is relevant when there is a risk for bleeding perioperatively but also an increased risk for postoperative thrombosis. For example this may be valid for patients with previous venous thromboembolism or stroke undergoing surgery.

The substance with fibrinogen-like activity is normally administered in the form of a pharmaceutical composition. Such a composition may contain the substance with fibrinogen-like activity in powder form such as, e.g., lyophilised powder, intended to be reconstituted with an aqueous medium before administration. An example of a suitable composition is the product Haemocomplettan P from Behring. This product contains human fibrinogen in pasteurized form as lyophilized powder and 1 g of the powder must be dissolved in 50 ml water for injection before administration. According to the manufacturer's information Haemocomplettan P 1 g contains 2025-3208 mg lyophilized powder, min. 1000 mg of human fibrinogen and the total content of protein is 1400-2000 mg. Haemocomplettan P 2 g contains 4050-6416 mg lyophilized powder, min. 2000 mg of human fibrinogen and the total content of protein is 2800-4000 mg. The powder also contains human albumin (the 1 g product: 400-700 mg) and substances adjusting the tonicity of the final composition (e.g. sodium chloride), adjusting the pH e.g. for stability issues during the lyophilisation process, for solubility reasons and/or for adjusting the pH to a value acceptable for injection purposes (e.g. sodium citrate—in the actual composition in the form of sodium citrate dihydrate). Moreover, the powder contains L-arginin hydrochlorid, which is contemplated to facilitating the lyophilisation process and/or together with sodium chloride and/or sodium citrate adjust the technical properties of the composition. Other salts may also be present such as a pharmaceutically acceptable salt like a phosphate, a carbonate, an amino acid salt including a lysinate or a glycinate, a salt of a carboxylic acid including an acetate, a butyrate, a valerate, a succinate, a hemisuccinate; a cyprionate or a trometamole salt or a combination thereof.

In line with the above discussion, a pharmaceutical composition may contain a powder comprising the substance with fibrinogen-like activity and optionally pharmaceutically acceptable excipients such as, e.g., pH adjusting agents, stabilizing agents, solubilizing agents, osmotic pressure adjusting agents, agents that reduce unspecific binding to proteins and/or lyophilising-facilitating or -stabilising agents.

Furthermore, the composition comprises an aqueous medium comprising water and optionally pharmaceutically acceptable excipients such as, e.g., pH adjusting agents, stabilizing agents, solubilizing agents, osmotic pressure adjusting agents and pharmaceutically acceptable salt (cf above).

A pharmaceutical composition containing the substance with fibrinogen-like activity is intended for parenteral administration, notably intraveneous administration by injection or infusion. Accordingly, the composition must fulfill any requirement with respect to sterility as defined e.g. in Ph.Eur. and USP.

Due to stability problems the substance with fibrinogen-like activity is dissolved in an aqueous medium just before use. The dissolution may be facilitated by heating to at the most 37° C. for at the most 15 minutes. However, it cannot be excluded that future developments enable manufacturing of storage-stable pharmaceutical compostions, i.e. compositions that are ready to use without any reconstitution step immediately before use. Such future compositions are also encompassed within the scope of the present invention.

### Other Aspects of the Invention

All particular and details outline above for the method aspect apply mutatis mutandis for the other following aspects of the invention.

Another aspect of the invention provides a kit comprising a substance with fibrinogen-like activity, an aqueous medium and instructions for using the kit in a method for preventing perk or postoperative bleeding as described herein above in the method aspect. The kit may comprise two separate containers, one comprising the substance with fibrinogen-like activity optionally together with one or more pharmaceutically acceptable excipients as described above and another container comprising an aqueous medium comprising water optionally together with one or more pharmaceutically acceptable excipients as described above, the kit furthermore comprising means with instructions for use of the kit for preventing perk and postoperative bleeding. In a specific embodiment the kit may comprise a substance with fibrinogen-like activity, a pharmaceutical acceptable carrier, and a device for administering the substance with fibrinogen-like activity, wherein the substance with fibrinogen-like activity is present in an amount effective for treating a subject in one or more administrations. The substance with fibrinogen-like activity may be in a dry or lyophilized form. Alternatively the substance with fibrinogen-like activity may be in a solution. Furthermore the kit may include one or more stabilizing agents along with the substance with fibrinogen-like activity. The kit may also include a device suitable for intravenous administration. In one embodiment the kit may additionally contain means for measuring the fibrinogen level.

The finding of fibrinogen as a biomarker for the risk of perk and postoperative bleeding also leads to the following further aspects of the invention, particularly in subjects with a preoperative fibrinogen plasma level in normal range, as defined above.

The invention also provides a method for prevention of perioperative and/or postoperative bleeding, the method comprising
i) sampling a biological sample from a subject,
ii) measuring fibrinogen content in the sample, and
iii) comparing the value obtained in step ii) with the normal level.

Furthermore, the invention provides a method for evaluating the risk of perioperative and/or postoperative bleeding, the method comprising
i) sampling a biological sample from a subject,
ii) measuring fibrinogen content in the sample, and
iii) identifying the risk of blood transfusion by use of a plot of plasma fibrinogen level versus risk.

Moreover, the present invention provides a method for evaluating the need for blood or plasma transfusion to a subject expected to undergo a surgical procedure, the method comprising
i) sampling a biological sample from a subject,
ii) measuring fibrinogen content in the sample, and
iii) comparing the value obtained in step ii) with the normal level.

In the above-mentioned methods, the biological sample is typically a blood, plasma or serum sample.

The subject is a mammal such as a human, notably the subject is expected to undergo a surgical procedure such as one of those mentioned hereinbefore. The subject is typically a subject with a preoperative fibrinogen plasma level in normal range or above, as defined above. Further, the above-mentioned methods also relate to a subject that has not received any therapeutic fibrinogen substitution or fibrinogen replacement therapy prior to the evaluation or prevention, such as e.g. 1 week, as defined above.

The above-mentioned methods are suitable for diagnosing the need for fibrinogen therapy using the measured value obtained in step ii) as an indicator, as explained hereinbefore.

In line with the above discussion, the present invention also relates to i) the use of plasma fibrinogen level as a biomarker for the prevention of perioperative and/or postoperative bleeding in a subject undergoing a surgical procedure. ii) the use of plasma fibrinogen level to predict the risk of perioperative and/or postoperative bleeding in a subject undergoing a surgical procedure, iii) the use of plasma fibrinogen level to predict the need for plasma or blood transfusion in a subject undergoing a surgical procedure.

To this end, the risk curve provided in FIG. 3 can be used as a guideline.

In a further aspect, the invention relates to the use of a substance with fibrinogen-like activity for the manufacture of a pharmaceutical composition for the prevention of perioperative and/or postoperative bleeding in a subject as described herein above.

The invention is further illustrated in the non-limiting examples and figures.

### LEGENDS TO FIGURES

FIG. 1 Correlation between preoperative fibrinogen concentration and postoperative bleeding in ten patients undergoing off-pump coronary artery bypass surgery. There was a significant inverse correlation between the two variables (r=−0.76, p<0.001).

FIG. 2 Correlation between preoperative fibrinogen concentration and postoperative bleeding in 170 patients undergoing coronary artery bypass surgery. There was a significant inverse correlation between the two variables (r=−0.53, p<0.001).

FIG. 3 Absolute risk for blood transfusion related to preoperative fibrinogen plasma concentration and gender.

FIG. 4 Fibrinogen plasma concentration at baseline, after fibrinogen infusion (in the Fib group), 2 h postoperatively and 24 h postoperatively. There was a significant difference between the two groups after infusion (p<0.01).

FIG. 5 Postoperative bleeding in the Fib group and in controls (mean±standard deviation). There was a significant difference between the two groups (p=0.012).

FIG. 6 Hemoglobin concentration at baseline, after fibrinogen infusion (in the Fib group), 2 h postoperatively and 24 h postoperatively. There was a significant difference between the two groups 24 h after surgery (p<0.05).

FIG. 7 Screening markers for hemostasis at baseline, after fibrinogen infusion (in the Fib group), 2 h postoperatively and 24 h postoperatively. There were no significant differences between the two groups at any time point.

FIG. 8 Variables reflecting coagulation at baseline, after fibrinogen infusion (in the Fib group), 2 h postoperatively and 24 h postoperatively. There were no significant differences between the two groups at any time point.

FIG. 9 Correlation between preoperative fibrinogen concentration and postoperative bleeding in 11 patients undergoing scoliosis surgery. There was a significant inverse correlation between preoperative fibrinogen concentration and perioperative bleeding, r=−0.63, p=0.039.

### Example 1

### Fibrinogen as a Selected Marker for Bleeding after Uncomplicated Off Pump Coronary Artery Bypass Surgery (OPCAB)

One common and important complication after cardiac surgery is bleeding. Bleeding may be caused by surgical factors or an impaired hemostasis, or a combination of both. Impaired post operative hemostasis may in turn be caused by different factors such as preoperative medication, underlying co-morbidities and the use of cardiopulmonary bypass (CPB). The aim of the study in this example was to investigate the association between selected markers of inflammatory activity, hemostasis and bleeding after uncomplicated off pump coronary artery bypass surgery. However, as reported below the present inventors found a close relationship between preoperative and postoperative fibrinogen levels and postoperative bleeding, despite the fact that all patients had preoperative fibrinogen concentrations within the normal range (2.0-4.5 g/L). Only results relating to fibrinogen are reported in this example.

### Patients

Ten patients undergoing OPCAB were included in the study. There were nine men and one woman with a mean age of 65±2 years. Exclusion criteria were unstable angina, redo surgery, serum creatinine>130 μmol/L, NSAID and steroid medication, known bleeding disorder and left ventricular ejection fraction<30%. Pre- and postoperative patient data are presented below:

TABLE
Patient characteristics. Median and range

| Number of patients | 10 | |
|---|---|---|
| Age (years) | 65 | (49-77) |
| Gender (M/F) | 9/1 | |
| LVEF (%) | 65 | (30-85) |
| Cleveland Clinic risk score | 1 | (0-4) |
| Number of anastomoses | 1 | (1-3) |

Key:
M = male,
F = female,
LVEF = left ventricular ejection fraction

Anticoagulant treatment (aspirin, clopidogel) was withdrawn at least one week before surgery. Low molecular weight protein was not administered before surgery. The protocol was approved by the Research and Ethics Committee at Gothenburg University and informed consent was obtained from all patients.

### Study Design

Fibrinogen (and selected markers of inflammation and hemostasis) was measured before and immediately after surgery. Preoperative samples were collected just prior to induction of anesthesia. Postoperative blood samples were collected at the end of surgery before closing the chest. Bleeding during the first 18 postoperative hours was registered. Correlation calculations between markers of inflammation, hemostasis and bleeding were performed.

### Surgical Procedure

Anesthesia was induced with remifentanil (0.5-1 μg/kg/min), propofol (1.5-2.5 mg/kg) and pancuronium (0.1 mg/kg) in all patients. The patients received 100 U heparin/kg bodyweight. Activated clotting time was aimed at >200 seconds. The heparin effect was not actively reversed after surgery. The patients were operated as reported previously (Wiklund et al. Scand Cardiovasc J. 2000; 34:197-200). In short, body temperature was kept at minimum 36° C. with the aid of elevated room temperature and a warming blanket. A generous crystalloid fluid regimen was kept as a mean to maintain a sufficient filling of the heart during manipulation and grafting. To achieve a bloodless field an intracoronary shunt (Flo-Thru-Shunt, Bio-Vascular Inc., St. Paul, Minn.) was used.

### Laboratory Analyses

Plasma fibrinogen was analysed with standard laboratory method.

### Statistical Analyses

The non-parametric Wilconxon's paired test was used to compare pre- and postoperative values within the group. Correlation was analyzed with Spearman Rank Sum Test. Statistical significance was defined as p<0.05. All the results are expressed as the median and the range.

### Results

All patients had an uncomplicated postoperative course and were discharged from hospital within 7 days.

### Bleeding

Median postoperative bleeding during the first 18 hours was 900 mL (190-940 mL).

### Hemostatic Variables

Anti-thrombin and fibrinogen levels decreased and β-thromboglobulin increased significantly after surgery.

TABLE
Hemostatic variables before and after surgery. Median and range

| | Preoperatively | | Postoperatively | | p value |
|---|---|---|---|---|---|
| Platelet count | 175 | (134-333) | 177 | (131-271) | 0.11 |
| (×10E9/L) | | | | | |
| β-thromboglobulin | 21.7 | (11.5-62.9) | 33.9 | (20.7-115.8) | 0.007 |
| (IU/mL) | | | | | |
| Anti-thrombin | 0.82 | (0.75-1.05) | 0.80 | (0.68-0.97) | 0.017 |
| (IE/mL) | | | | | |
| D-Dimer (mg/L) | 0.32 | (0.09-1-41) | 0.28 | (0.10-1.69) | 0.51 |
| Fibrinogen (g/L) | 2.46 | (1.85-3.90) | 2.36 | (1.65-3.57) | 0.012 |

### Correlation Between Fibrinogen Level and Bleeding

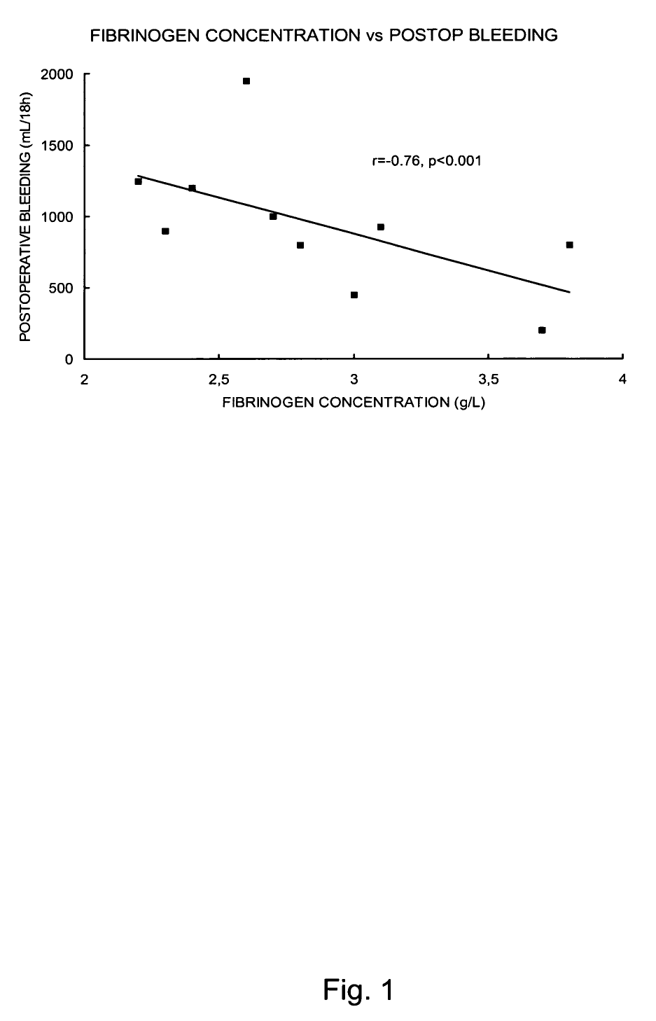

There were significant inverse correlations between postoperative bleeding and pre- and postoperative fibrinogen levels (preoperative: r=−0.76, p=0.011; postoperative: r=−0.84, p=0.002). FIG. 1 shows the correlation between preoperative fibrinogen level and postoperative bleeding.

### Discussion

As seen from the table above, all patients had fibrinogen levels within the normal range both before and after surgery. However, the results indicate that preoperative fibrinogen analysis may provide important information about the risk for postoperative bleeding and may be controlled at least in patients with increased risk for bleeding. Moreover, even individuals with low levels (within the normal range) may benefit from perioperativ fibrinogen therapy.

### Example 2

### Fibrinogen, a Potential Biomarker for Bleeding and Blood Transfusion after Cardiac Surgery

Based on the results obtained in Example 1, a prospective descriptive study was carried out including 170 patients operated with cardiopulmonary bypass. As described in the following only preoperative fibrinogen concentration was an independent predictor of postoperative bleeding. The results indicate that preoperative fibrinogen concentration (even within the normal range) is a limiting factor for postoperative hemostasis. Preoperative management of fibrinogen concentration provides information about the risk for extensive bleeding and blood transfusion after cardiac surgery.

### Patients

Initially 175 consecutive patients (mean age 67 years, 75% males) undergoing first time elective coronary artery bypass grafting (CABG) were included in the study. Exclusion criteria were acute CABG, known hepatic disorder, known bleeding disorder and surgical bleeding at re-exploration. Five patients were excluded due to surgical bleeding resulting in 170 patients finally included. 151 patients (89%) had ongoing treatment with aspirin at the time of surgery, 33 patients (19%) had been treated with clopidogrel before surgery, 54 patients (32%) with low molecular weight-heparin (LMWH), and six patients (3.5%) with warfarin.

TABLE
Patients included in the study

| | |
|---|---|
| n | 170 |
| Mean age (years) | 68 ± 9.4 |
| Male gender (%) | 75 |
| Unstable angina (%) | 55 |
| CPB time (min) | 73 ± 24 |
| Aastomoses (n) | 3.0 ± 0.8 |
| Preoperative medication (%) | |
| ASA (acetylsalicylic acid) | 89% |
| LMW heparin | 32% |
| Clopidogrel | 19% |
| Warfarin | 3.5% |

### Clinical Management

The anesthesia in all patients was induced with 200-300 μg of fentanyl and 3-5 μg of thiopental followed by pancuronium 0.1 mg/kg. Anesthesia was maintained with sevoflurane, and propofol was used during cardiopulmonary bypass (CPB). The patients received 300 units of heparin/kg bodyweight in order to maintain an activated clotting time (ACT) at >480 seconds. After the surgery was performed, the ACT was reversed by protamin administration (1 mg protamin/100 U heparin) to an ACT of <130 seconds.

The CPB circuit included a membrane oxygenator and roller pumps. Standard nonpulsatile CBP technique with moderate hypothermia (bladder temperature 34-35° C.) and hemodilution was used. Cardioprotection was achieved with antegrade, cold blood cardioplegia. Weaning off CBP was performed after rewarming to a bladder temperature of at least 36° C.

Aspirin therapy was not discontinued before surgery. Low molecular weight heparin was discontinued 12 hours before surgery, clopidogrel and warfarin five days before surgery. All patients received 2 g tranexamic acid before anesthesia induction and 2 g immediately after skin closure. Aprotinin was not used in any of the study patients.

### Study Design and Analyses

The association between bleeding and blood transfusion, and the following pre- and postoperative variables were investigated: age, gender, body mass index (BMI), number of grafts, unstable angina, extracorporal circulation time, aortic clamp time, anticoagulation therapy, plasma fibrinogen, platelet count, APTT (activated partial thromboplastin time), and PT (prothrombin time).

Plasma fibrinogen, hemoglobin, platelet count, APTT and PT were analyzed the day before surgery with standard clinical methods. Five mL of blood was collected by antecubital veni-puncture in tubes containing 0.5 ml, 0.13 mmol/L sodium citrate and centrifuged at room temperature for 20 min at 2200 g. The plasma fibrinogen concentration was determined according to the method by Clauss (Clauss, Acta Haematol 1957; 17: 237-46) with the use of an assay in which excess thrombin is added to diluted, low fibrinogen containing plasma, in order to determine the amount of clottable protein (STA-R®, Diagnostica Stago, Asnieres, France). Reference value is 2.0-4.5 g/L (normal range). APTT was analyzed with a clotting method in which platelet poor plasma is incubated with APTT-reagent (STA-PTT® Automate 5, Diagnostica Stago, Asnieres, France). Reference value for adults is 30-42 seconds. Prothrombin complex was analyzed by a standard clotting method according to the reference method by Korsan-Bengtsen (Korsan-Bengtsen, Scand J Haematol 1971; 8: 369-74) (Stago Prothrombinkomplex Assay SPA 50®, Diagnostica Stago, Asnieres, France) and reported as International Normalized Ratio (INR). Reference value for adults is <1.2 INR. INR is the ratio of the patient's prothrombin time and the prothrombin time of normal reference plasma.

By the same veni-puncture, 3 mL of blood was drawn into tubes containing ethylene diamine tetracetate for analysis of hemoglobin and platelet counts. The plasma hemoglobin concentration was measured by photo spectrometry at 540 nm (Cell-Dyn® 4000 system, Abbott, St Clara, Calif., USA). Reference value is for males 134-170 g/L, and for females 117-153 g/L. Platelet counts were performed by flow cytometry (Cell-Dyn® 4000 system, Abbott, St Clara, Calif., USA). Reference value for males is 145-348×10⁹/L and for females 165-387×10⁹/L.

Postoperatively, the total amount of chest tube drainage after closure of the sternum and during the first 12 postoperative hours was registered. In cases of re-exploration, the bleeding volume until re-exploration was registered. A surgical bleeding was established at the time of re-exploration if a specific site/sites was identified which could be controlled by surgical means. Patients with surgical bleeding (n=5) were excluded from further analysis. The amount of transfused blood products (red blood cells, fresh frozen plasma, and platelets) during the hospital stay was recorded. Patients were transfused if the following criteria were met: 1) a blood hemoglobin level<80 g/L and/or a central venous oxygen saturation<60%, and/or 2) hemodynamic instability. Unstable angina was defined according to the Braunwald classification (Braunwald E. Unstable angina. A classification. Circulation 1989 80(2):410-4).

### Statistics

Results are expressed as mean and standard deviation (SD) or number and percent (%).

Statistical significance was defined as a p-value<0.05. Independent-samples t-test was used to analyze for differences in clinical and hematological variables between patients who did and did not undergo re-sternotomy for diffuse bleeding. Simple linear regression was used to analyze the relationship between hematological and demographic data and the amount of postoperative bleeding. The following variables were included in the univariate analyses: age, gender, BMI, stable vs. unstable angina, number of grafts, extracorporeal circulation time, aortic clamp time, preoperative hemoglobin level, platelet count, APTT, PT and fibrinogen concentration. Linear multiple regression analysis using forward selection was then used to analyze the joint influence of the same variables. Since bleeding volume was positively skewed, univariate correlation and regression analysis was based on logarithmic bleeding values. Comparisons between transfused and non-transfused patients were done with two-sample t-tests for continuous data and with chi-square tests for categorical data. The risk of transfusion was analyzed with multiple logistic regression using the preoperative predictors above.

### Results

### Clinical Course

165 of the 170 patients had an uncomplicated postoperative course and were discharged from the hospital within seven days. One patient with a postoperative myocardial infarction developed heart failure and died of multi-organ failure nine days after surgery. Four of the 170 (2.3%) patients were re-explored within the first 12 hours due to extensive diffuse postoperative bleeding.

### Bleeding and Transfusions

Mean postoperative bleeding was 421±258 mL/12 h. In total 29 patients (17%) received transfusions of blood or blood products. Twenty patients received transfusions with packed red blood cells (RBC) only, three patients received RBC and plasma, one patient RBC and platelets, and two patients received transfusions with RBC, plasma and platelets. Two patients received plasma only and one patient plasma and platelets.

### Laboratory Variables

### Fibrinogen

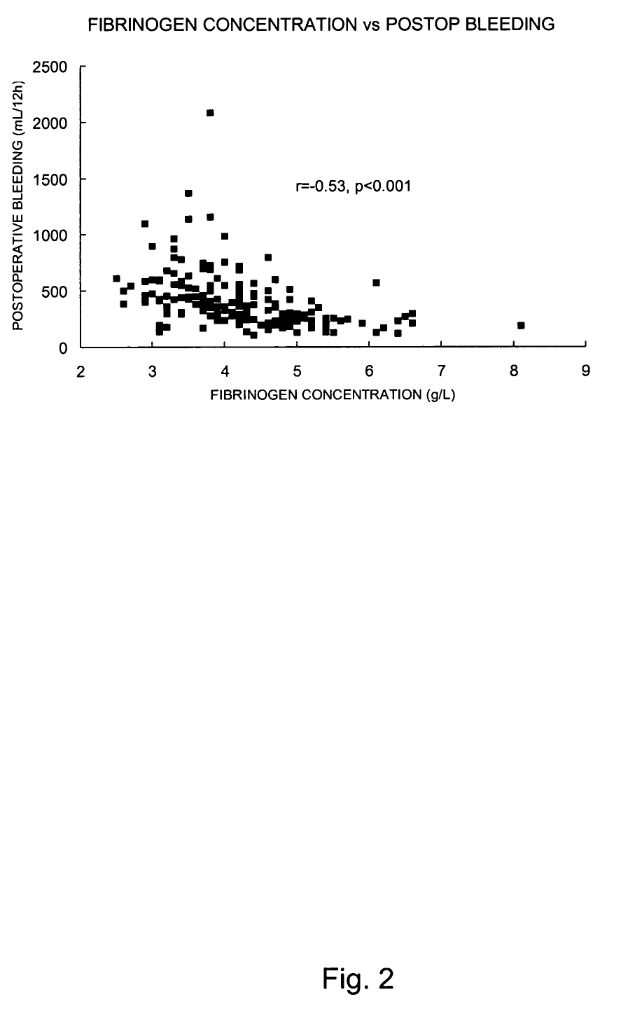

All patients had preoperative fibrinogen concentration over the lower normal limit (2.0 g/L). 116 patients (68%) had values within the normal range (2.0-4.5 g/L), and the remaining 54 patients (32%) had higher values. Mean fibrinogen concentration was 4.2±0.9 g/L, range 2.4-8.1 g/L, FIG. 2.

Patients undergoing re-exploration because of diffuse bleeding (n=4) had numerically but not statistically significant lower preoperative fibrinogen levels compared to the not re-operated patients (3.6±0.2 vs. 4.2±0.9 g/L p=0.17).

### Platelet Count, Hb, APTT and PT

The mean preoperative platelet counts, APTT and PT values were all within the normal range, except for six patients who had slightly elevated PT (INR 1.3-1.9). Five of these had been on warfarin therapy until five days before surgery. The mean preoperative hemoglobin concentration was 140±14 g/L, which postoperatively was reduced to 114±13 g/L on day one and 105±11 g/L on day two after surgery.

Association between Pre- and Peroperative Variables, Bleeding and Blood Transfusions Bleeding

There were no statistically significant differences in bleeding volumes between patients in relation to their preoperative anti-coagulation regimens: no medication (393±318 mL, n=9), only aspirin (473±289 mL, n=78), only clopidogrel (390±220 mL, n=3), only LMWH (318±25 mL, n=2), only warfarin (365±152 mL, n=4), aspirin+clopidogrel (361±164 mL, n=20), aspirin+LMWH (362±222 mL, n=37), clopidogrel+LMWH (305 mL, n=1) aspirin+warfarin (508±152 mL, n=2) and aspirin+LMWH+clopidogrel (434±289 mL, n=14).

The univariate correlations between pre- and perioperative variables and postoperative bleeding are given in the following table.

TABLE
Univariate correlation between clinical and laboratory
variables and log bleeding after CABG.

| | r | p-value |
|---|---|---|
| Age (years) | 0.09 | 0.24 |
| Gender | 0.14 | 0.08 |
| BMI (kg/m²) | −0.15 | 0.06 |
| Unstable angina | −0.009 | 0.90 |
| Preoperative medication | | |
| Aspirin | 0.09 | 0.27 |
| LMWH | 0.13 | 0.09 |
| Clopidogrel | −0.02 | 0.76 |
| Warfarin | 0.006 | 0.94 |
| Hemoglobin (g/L) | −0.25 | 0.001 |
| Platelet Count (×10⁹/L) | −0.26 | 0.001 |
| APTT (s) | 0.07 | 0.37 |
| PT (INR) | 0.05 | 0.49 |
| Fibrinogen (g/L) | −0.53 | <0.0001 |
| Anastomoses (n) | 0.11 | 0.16 |
| ECC (min) | 0.06 | 0.47 |
| Aortic Clamp Time (min) | 0.08 | 0.28 |

Key:
APTT = Activated partial thromboplastin time,
BMI = Body mass index,
ECC = Extracorporeal circulation,
Hb = Hemoglobin,
INR = International normalized ratio,
LMWH = Low molecular weight heparin,
PT = Prothrombin time.

There were significant inverse correlations between log postoperative bleeding and preoperative fibrinogen concentration (r=−0.53, p<0.001, FIG. 2), preoperative platelet count (r=−0.26, p=0.001) and preoperative haemoglobin concentration (r=−0.25, p=0.001). Neither APTT (r=0.07, p=0.37), nor PT (r=0.05, p=0.49) or preoperative anticoagulation did correlate to bleeding,

In multivariate testing preoperative fibrinogen concentration was the only independent predictor of postoperative bleeding (r=−0.53, p<0.001).

### Blood Transfusions

Pre- and perioperative variables for patients receiving blood transfusions and those not receiving transfusion are given in the following table.

TABLE
Pre- and peroperative variables in transfused
and non-transfused patients.

| | Transfused | Non-transfused | |
|---|---|---|---|
| | (n = 29) | (n = 141) | p |
| Age (years) | 70 ± 10 | 66 ± 9 | 0.052 |
| Male gender | 16 (55%) | 112 (79%) | 0.006 |
| BMI (kg/m²) | 26 ± 5 | 27 ± 4 | 0.17 |
| Unstable angina | 18 (62%) | 76 (54%) | 0.42 |
| Preoperative medication | | | |
| Aspirin | 26 (90%) | 125 (89%) | 0.88 |
| LMWH | 6 (21%) | 48 (34%) | 0.16 |
| Clopidogrel | 10 (34%) | 42 (30%) | 0.62 |
| Warfarin | 1 (3%) | 8 (6%) | 0.63 |
| Hemoglobin (g/L) | 136 ± 14 | 141 ± 14 | 0.041 |
| Platelet Count (×10⁹/L) | 255 ± 66 | 267 ± 71 | 0.40 |
| APTT (s) | 39 ± 9 | 37 ± 5 | 0.15 |
| PT (INR) | 1.0 ± 0.2 | 1.0 ± 0.1 | 0.47 |
| Fibrinogen (g/L) | 3.9 ± 0.8 | 4.3 ± 0.9 | 0.08 |
| Anastomoses (n) | 3.2 ± 0.8 | 3.0 ± 0.8 | 0.11 |
| ECC (min) | 82 ± 26 | 71 ± 23 | 0.023 |
| Aortic Clamp Time (min) | 49 ± 16 | 41 ± 14 | 0.009 |

Mean ± SD or number (percentage).
Key:
APTT = Activated partial thromboplastin time,
BMI = Body mass index,
ECC = Extracorporeal circulation,
INR = International normalized ratio,
LMWH = Low molecular weight heparin,
PT = Prothrombin time,
SD = Standard deviation

Transfused patients were more often female (45% vs 21%, p=0.006), had longer ECC time (82±26 vs 71±23 min, p=0.023), longer aortic clamp time (49±16 vs 41±14, p=0.009), and lower preoperative haemoglobin concentration (136±14 vs 141±14 g/L, p=0.041), while preoperative fibrinogen concentration tended to be lower (3.9±0.8 vs 4.3±0.9 g/L, p=0.08). In contrast, there were no statistical differences in age, BMI, number of grafts, incidence of unstable angina, preoperative anticoagulation, or preoperative platelet count, PT or APTT.

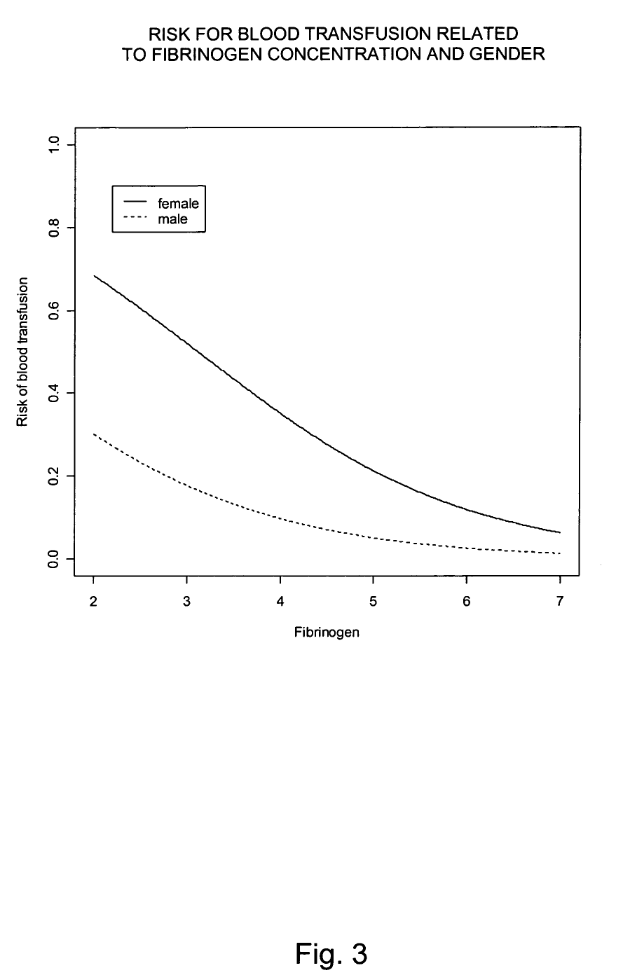

Independent predictors of blood transfusion were in a logistic regression model: preoperative fibrinogen concentration (OR=2.0 (95% confidence interval 1.1-3.7) per one g/L decrease, p=0.027), female gender (OR=5.0 (1.8-14.7), p=0.002) and aortic cross clamp time (OR=1.03 (1.01-1.06) per minute, p=0.013). The risk for blood transfusion for men and women with different preoperative fibrinogen concentrations are given in FIG. 3.

### Discussion

The main finding in the present study was that preoperative fibrinogen plasma concentration is an independent predictor of postoperative bleeding and blood transfusion after coronary artery bypass surgery. All patients had preoperative fibrinogen levels over the normal lower limit and the results suggest therefore that 1. Fibrinogen level, even within the normal range, is a limiting factor for postoperative hemostasis. 2. Fibrinogen may be used as a biomarker to identify patients with an increased risk for excessive bleeding and blood transfusion after cardiac surgery.

Cardiac surgery and the use of CPB induce substantial alterations in the hemostatic system. The explicit role of preoperative fibrinogen level as a predictor of postoperative bleeding has been investigated in a few studies with conflicting results. Some authors have found a weak correlation while others have not been able to show any association (Gravlee G P, et al. Predictive value of blood clotting tests in cardiac surgical patients. Ann Thorac Surg 1994; 58:216-21, Hall T S, et al. Hemorrhage related reexploration following open heart surgery: the impact of pre-operative and post-operative coagulation testing. Cardiovasc Surg 2002; 10:146-53, Carroll R C et al, Correlation of perioperative platelet function and coagulation tests with bleeding after cardiopulmonary bypass surgery. J Lab Clin Med 2006; 147:197-204, Wahba A, et al. Predictors of blood loss after coronary artery bypass grafting. J Cardiothorac Vasc Anesth 1997; 11:824-7, Nuttall G A et al. Coagulation tests predict bleeding after cardiopulmonary bypass. J Cardiothorac Vasc Anesth 1997; 11:815-23, Khuri S F et al. Hematologic changes during and after cardiopulmonary bypass and their relationship to the bleeding time and nonsurgical blood loss. J Thorac Cardiovasc Surg 1992; 104:94-107, Fassin W et al. Predictive value of coagulation testing in cardiopulmonary bypass surgery. Acta Anaesthesiol Belg 1991; 42:191-8).

However, it is difficult to compare the previous studies due to variations in patient selection and study design. The present study was designed exclusively to study the association between preoperatively measured plasma fibrinogen, and the amount of postoperative bleeding and blood transfusions in a prospective manner. The design was standardized by measuring fibrinogen the day before study rather than after arrival in the operating theatre, at which time point fibrinogen concentrations in plasma might be affected by preoperative fluid replacement. Bleeding was handled as a continuous variable in contrast to the majority of previous studies where patients have been divided into two groups, bleeders and non-bleeders with different definitions and limits. Furthermore, bleeding was registered during the first 12 hours postoperatively instead of bleeding until the next morning or until the drains were withdrawn. It is possible that this strict design explains why the correlation between preoperative fibrinogen was higher in the present study than previously reported.

Plasma fibrinogen level is dependent upon both genetic and environmental factors. Current evidence suggests that plasma fibrinogen levels are probably under substantial genetic control, since genetic polymorphisms account for some 20-51% of variations in plasma fibrinogen levels (Green F R. Fibrinogen polymorphisms and atherothrombotic disease. Annals of the New York Academy of Sciences 2001; 936:549-59). Environmental factors related to plasma fibrinogen concentration are e.g. age, body mass index, and diabetes (Humphries S E, Henry J A, Montgomery H E. Gene-environment interaction in the determination of levels of haemostatic variables involved in thrombosis and fibrinolysis. Blood Coagul Fibrinolysis 1999; 10:S17-21). In the present study we found a significant correlation between plasma fibrinogen level and postoperative bleeding, FIG. 2. In contrast, no correlation was detected between bleeding and standard test for coagulopathy (APTT and PT). A significant inverse correlation was detected between bleeding and platelet count. However, the correlation between platelet count and bleeding (r=−0.26) was considerable less pronounced than between fibrinogen and bleeding (r=−0.53) and in the multivariate test only fibrinogen remained as independent predictor. The results demonstrate thus that the patient's preoperative fibrinogen level provide more information about risk for postoperative bleeding compared to standard clinical screening tests for coagulopathy.

The association between preoperative fibrinogen concentration and blood transfusions in the present study has to the best of our knowledge not been reported before. This finding is central; it extends the importance of the statistical correlation between fibrinogen and bleeding further into clinical relevance, since blood transfusions are associated with increased morbidity and mortality, immunoactivation and risk for transfer of pathogens (Murphy G J, Angelini G D. Indications for blood transfusion in cardiac surgery. Ann Thorac Surg 2006; 82:2323-34, Spiess B D. Transfusion of blood products affects outcome in cardiac surgery. Semin Cardiothorac Vasc Anesth 2004; 8:267-81). Gender was also a significant predictor for blood transfusion in the present study, which confirms previous observations (Dial S, Delabays E, Albert M, Gonzalez A, Camarda J, Law A, et al. Hemodilution and surgical hemostasis contribute significantly to transfusion requirements in patients undergoing coronary artery bypass. J Thorac Cardiovasc Surg 2005; 130654-61, Ferraris V A, Gildengorin V. Predictors of excessive blood use after coronary artery bypass grafting. A multivariate analysis. J Thorac Cardiovasc Surg 1989; 98:492-7. The association between gender, fibrinogen concentration and risk for blood transfusion is given in FIG. 3.

Fibrinogen concentrate is commercially available and used to treat patients with inherited or acquired hypo-, dys- or afibrinogenemia, and as rescue therapy in patients with on-going massive bleeding (Bolton-Maggs P H, Perry D J, Chalmers E A, Parapia L A, Wilde J T, Williams M D, et al. The rare coagulation disorders—review with guidelines for management from the United Kingdom Haemophilia Centre Doctors' Organisation. Haemophilia 2004; 10:593-628, Ketchum L, Hess J R, Hiippala S. Indications for early fresh frozen plasma, cryoprecipitate, and platelet transfusion in trauma. The Journal of Trauma 2006; 60:S51-8). Based on the present results, one may speculate that prophylactic preoperative fibrinogen substitution may be an approach to reduce bleeding and blood transfusions in patients with low fibrinogen levels undergoing cardiac surgery. However, this concept needs to be investigated in future studies, which also need to explore the risk of early graft occlusion rate when the hemostasis is artificially altered.

In summary, the results suggest that the preoperative fibrinogen concentration in plasma, even within the normal range, is a limiting factor for postoperative hemostasis. Preoperative fibrinogen analysis provides additional information about risk for excessive postoperative bleeding and may be measured, at least in patients with other risk factors for increased bleeding.

### Example 3

### Fibrinogen Concentrate Infusion in Cardiac Surgery Patients

The aim of the present study was to investigate the effect of administration of fibrinogen to patients undergoing elective coronary artery bypass grafting. All patients had preoperative levels of plasma fibrinogen in the low normal range (<3.8 g/L).

### Patients

20 patients were included in the study; 10 in the fibrinogen group (FIB group) and 10 in the control group (randomized study). The patient characteristics were as follows:

| | FIB group | Control group |
|---|---|---|
| n | 10 | 10 |
| age (years) | 66 ± 9 | 68 ± 8 |
| Gender (M/F) | 9/1 | 9/1 |
| ECC time (min) | 73 ± 26 | 70 ± 24 |
| Anastomoses (n) | 2.9 ± 0.7 | 2.9 ± 0.9 |

Key: M = male,
F = female,
ECC = Extracorporeal Circulation

### Clinical Management

The clinical management of the patients were identical as in Example 2.

### Study Design

Prospective double-blind randomized study in 20 patients undergoing elective coronary artery bypass grafting with preoperative levels of plasma fibrinogen in the low normal range (<3.8 g/L). Exclusion criteria were surgical bleeding at acute re-exploration, known liver or renal disease, known bleeding disorder. Patients were randomized to infusion of 2 g fibrinogen (Haemocomplettan®) or nothing before surgery. Primary endpoint was safety (clinical adverse events and graft occlusion assessed by computer tomography scanning (CT) 3-4 days after surgery). Secondary endpoints were peroperative bleeding, bleeding<12 hours postoperatively, blood transfusions and effects of fibrinogen infusion on hemostatic laboratory variables (thromboelastography with INTEM, FIBTEM, HEPTEM and EXTEM, coagulation (antithrombin, thrombin-antithrombin complex, thrombin fragment 1 and 2), fibrinolysis (D-dimer) and platelet aggregability (Multiplate)). Laboratory measurements were performed at baseline (before surgery), after infusion (before surgery) and 2 hours and 24 hours after surgery.

The laboratory variables were determined as described in Example 2.

### Statistics

Continuous variables were compared between the groups with Mann-Whitney test. Categorical variables were compared with Chi-square test.

### Results

There were no differences in baseline pre- and perioperative variables between the two groups. Mean preoperative plasma fibrinogen concentration was 3.0±0.1 g/L (mean±SEM). Infusion of 2 g fibrinogen increased plasma fibrinogen concentration with 0.4±0.1 g/L (figures not corrected for hematocrit).

The patients' characteristics preoperatively were as follows:

| | FIB group | Control group |
|---|---|---|
| HB (g/L) | 150 ± 8 | 150 ± 10 |
| Platelet count (×10⁹/L) | 253 ± 67 | 257 ± 40 |
| APTT (s) | 36 ± 5 | 33 ± 2 |
| PT (INR) | 1.05 ± 0.07 | 1.03 ± 0.08 |
| Fibrinogen (g/L) | 3.3 ± 0.3 | 3.3 ± 0.4 |

Key:
HB = Hemoglobin,
APTT = Activated prothrombin time.
PT, Prothrombin time

All patients recovered normally after surgery and were discharged from the clinic within seven days. There were no clinically detectable adverse effects of fibrinogen infusion. All patients had a postoperative CT scan. There were totally 56 grafts (18 LIMA to KAD grafts) and 38 vein grafts. LIMA graft patency was 100% (18/18). There was one vein graft occlusion in the fibrinogen group and none in the control group resulting in a vein graft patency of 97% (37/38). One patient in the fibrinogen group had a subclinical pulmonary emboli detected co-incidentally by CT.

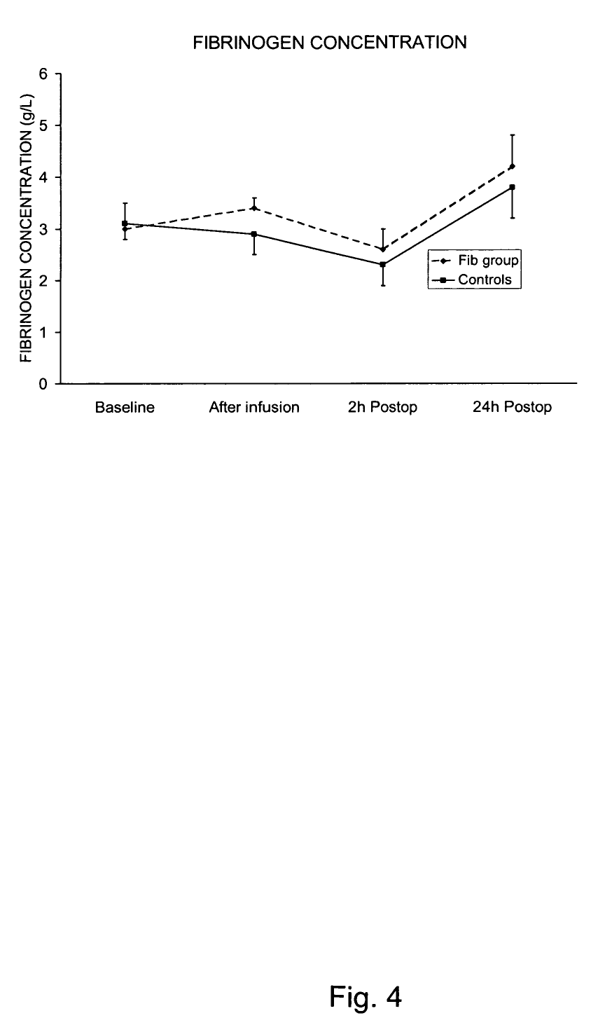

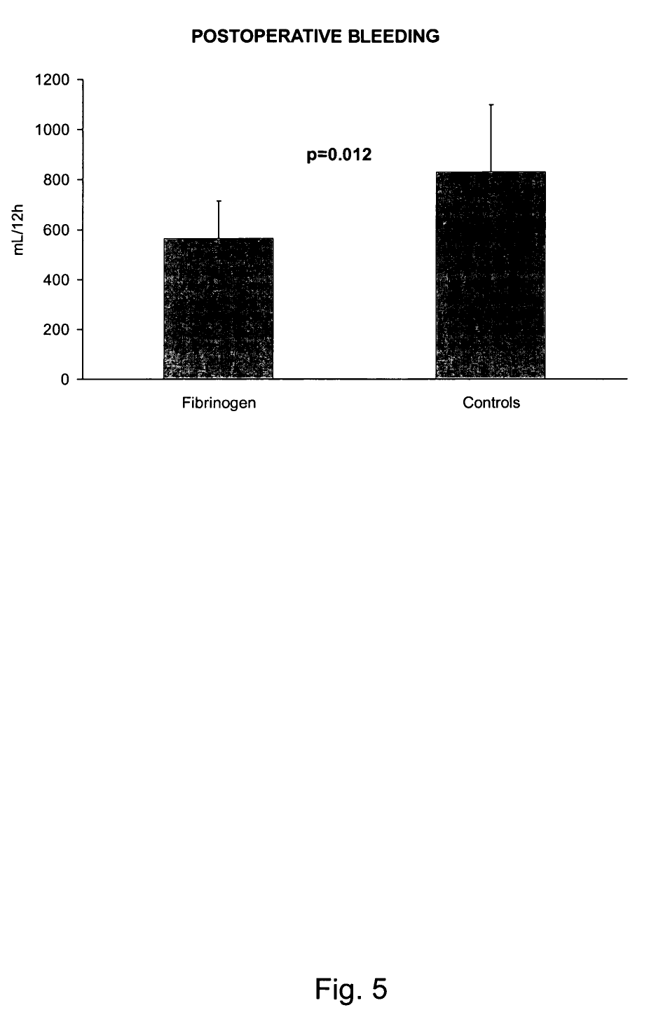

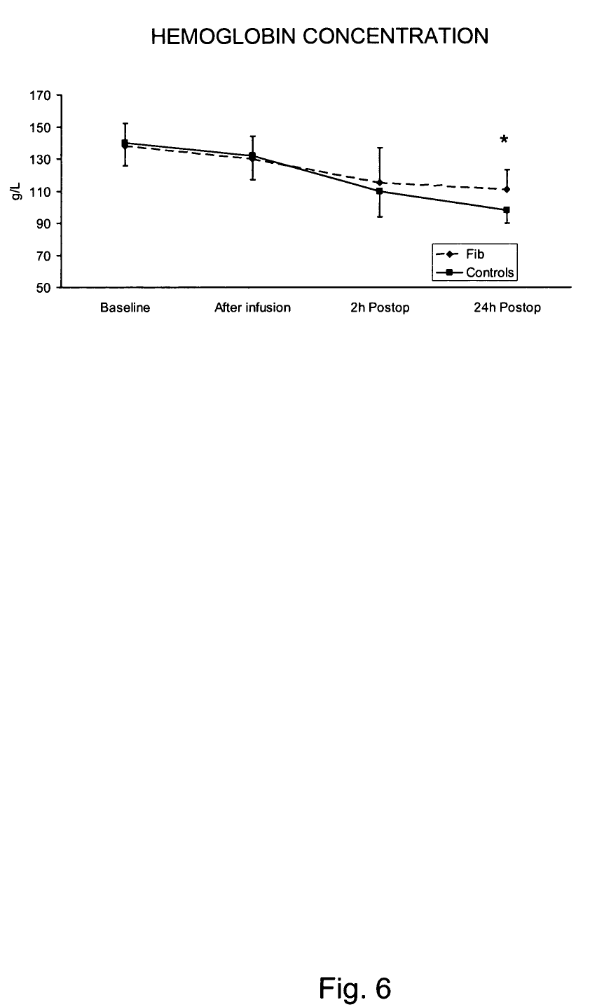

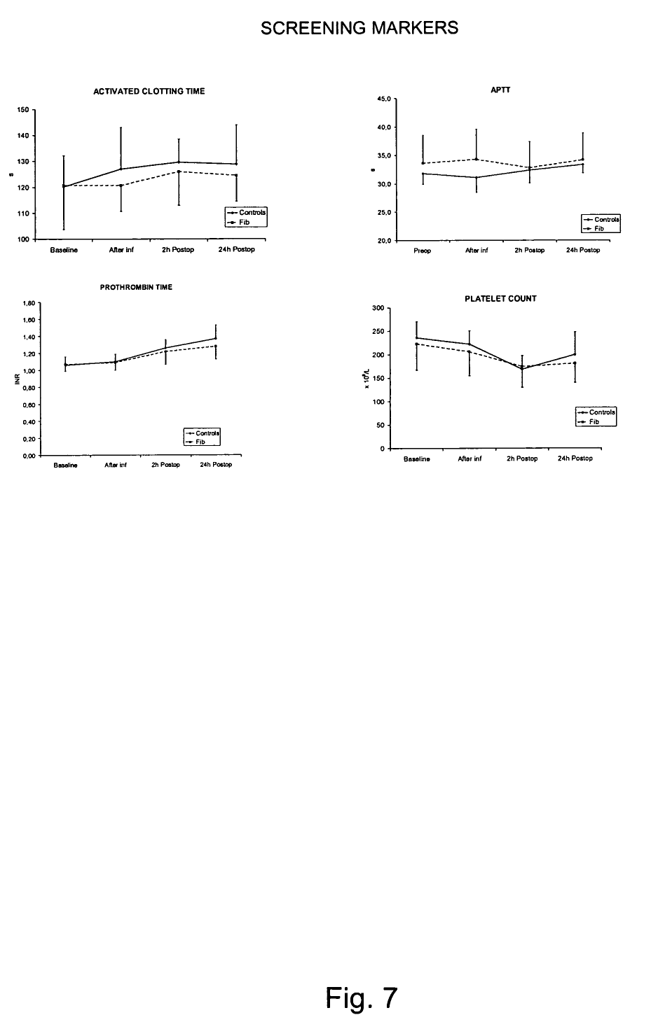

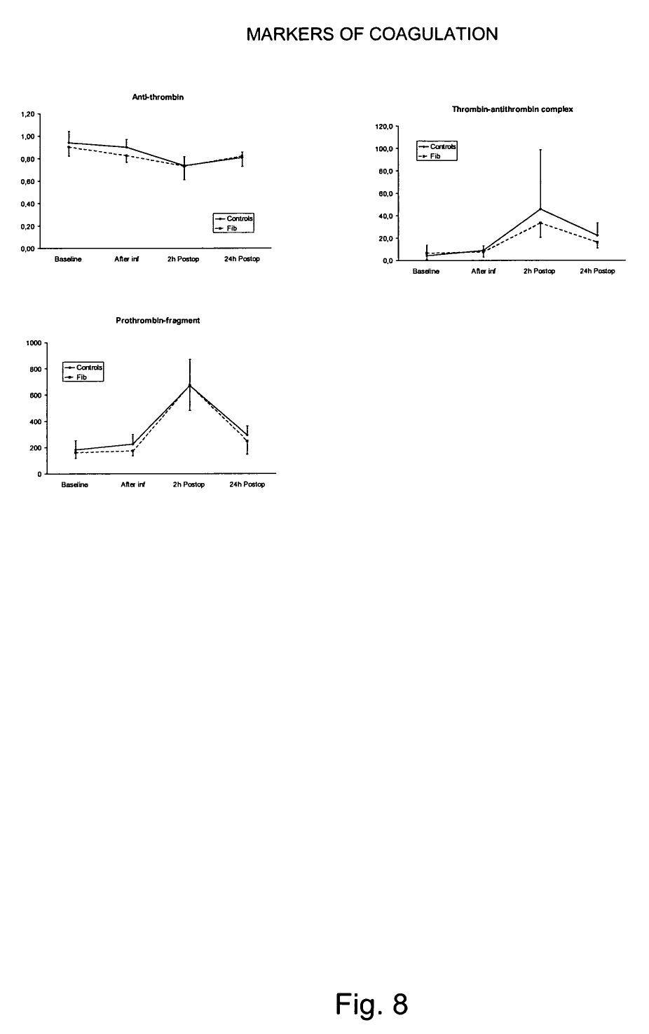

The results are shown in FIGS. 4-8. FIG. 4 shows the influence of fibrinogen infusion on plasma levels of fibrinogen in the patients. The fibrinogen levels were higher after infusion in the FIB group while postoperative levels did not differ significantly between the groups. FIG. 5 shows that the patients receiving fibrinogen had a significantly lower bleeding than the patients in the control group. These results are also supported by the results shown in FIG. 7, where the hemoglobin level 24 h after surgery is higher for the fibrinogen group than for the control group despite that three patients in the control group received blood transfusions. In addition, there were no effects on postoperative coagulation (FIGS. 7 and 8). Three patients in the control group and one patient in the FIB group received blood transfusions postoperatively.

### Conclusions

The present study has shown that administration of fibrinogen before surgery reduces postoperative bleeding and diminishes postoperative hemoglobin loss. No clinically detectable side effects were observed and no influence on postoperative the coagulation factors was observed.

Overall the examples show:

1. Preoperative measurement of fibrinogen can predict risk of bleeding and need for blood transfusion after cardiac surgery
2. Prophylactic treatment with fibrinogen reduces postoperative bleeding and diminishes postoperative haemoglobin deficiency

### Example 4

### Plasma Fibrinogen Level and Risk of Bleeding in Patients Undergoing Back Surgery

### Background

As reported in the clinical studies disclosed in examples 1-3, a significant correlation between preoperative plasma concentration of fibrinogen and postoperative bleeding in patients undergoing cardiac surgery has been demonstrated. The results indicate that the fibrinogen level is a limiting factor for postoperative bleeding. In the present study we sought to determine if this is a generalized mechanism, not only confined to cardiovascular surgery, by analyzing a potential correlation between fibrinogen levels and perioperative bleeding in back surgery patients.

### Methods

Eleven patients undergoing elective scoliosis surgery were included in a prospective descriptive study. Plasma concentration of fibrinogen was measured 7 to 14 days before surgery in all patients. Perioperative bleeding (from start of surgery until drains were removed (12 to 48 hours postoperatively) was registered. Correlation between the two variables was calculated with Spearman's Rank sum test.

### Results

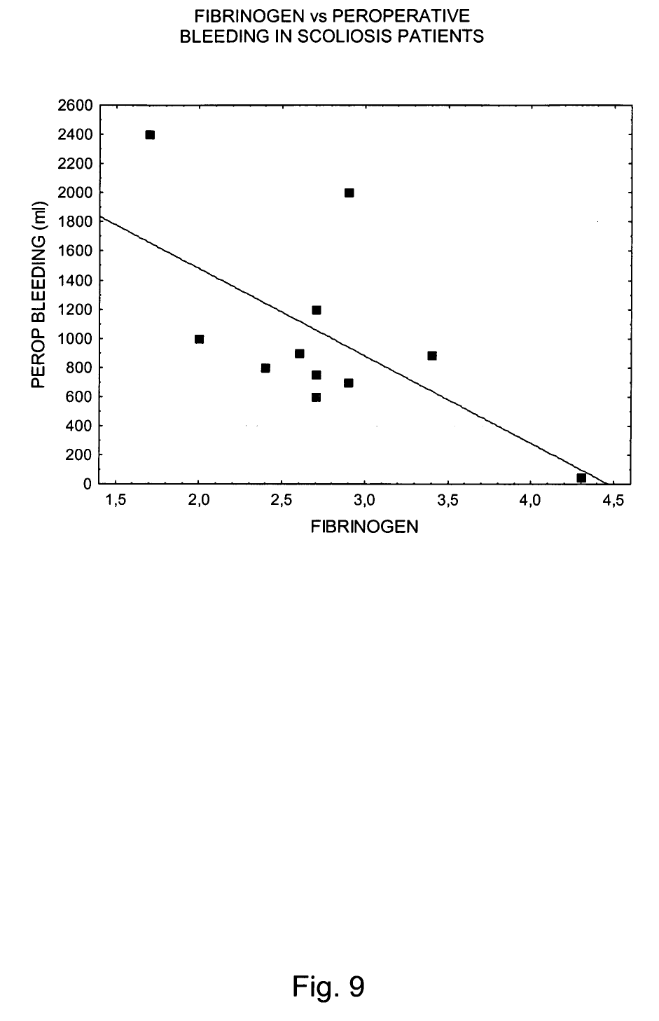

Ten of the eleven patients had fibrinogen concentration within the normal interval (2.0-4.5 g/L). One patient had a concentration of 1.7 g/L. Mean fibrinogen concentration was 2.7±0.7 g/L (range 1.7-4.3 g/L). Mean bleeding volume was 1030±653 ml (range 50-2400 ml). There was a significant inverse correlation between preoperative fibrinogen concentration and perioperative bleeding, r=−0.63, p=0.039, FIG. 9.

### Conclusions

The results suggest that the association between perioperative bleeding and plasma concentration of fibrinogen, previously demonstrated in cardiac surgery patients, is a generalized mechanism. Scoliosis patients with low fibrinogen concentrations may benefit from prophylactic fibrinogen treatment.

### Example 5

### Prophylactic Fibrinogen Concentrate Infusion in Cardiac Surgery

### Effect on Transfusion Requirements, Bleeding and Costs

In the studies disclosed in example 1 and 2 of the present invention, the inventors showed a surprisingly high inverse correlation between the preoperative concentration of fibrinogen in plasma and the volume of postoperative bleeding in two different patient populations (r=−0.76 and −0.53, respectively) undergoing coronary bypass surgery, CABG. Interestingly, all patients had a preoperative fibrinogen concentration above the lower normal limit (2.0 g/L). The results indicated further that preoperative fibrinogen concentration is an independent predictor of bleeding volume and blood transfusion after CABG. Taken together, the results of the clinical studies in example 1 and 2 suggested that fibrinogen is a limiting factor for postoperative hemostasis after cardiac surgery even within the normal range. However, infusion of coagulation factors before or during CABG may induce hypercoagulability with an increased risk of early graft occlusion and myocardial infarction. Before the efficacy of prophylactic fibrinogen infusion can be tested in larger patient populations, safety and tolerability needs to be evaluated in smaller patient groups. Therefore, as disclosed in example 3 herein, a prospective blinded randomized pilot study with 20 patients, were designed to also assess whether prophylactic infusion of fibrinogen concentrate in CABG patients is safe (clinical side effects and graft occlusion assessed by CT scan) and tolerable. In the clinical study disclosed in example 3, no clinically adverse events of fibrinogen infusion were detected. However, it was showed that fibrinogen concentrate infusion reduced intraoperative bleeding and postoperative bleeding. Accordingly, the next step in accordance with a standard clinical developmental programme is to confirm the findings in a “proof of concept” study (phase II), before entering the clinical phase 3 or “registration studies”. To further confirm that prophylactic fibrinogen can be used clinically to prevent bleeding and blood transfusion and to further confirm the safety and efficacy data from previously described pilot studies, the investigator has planned to conduct the following prospective double-blind placebo-controlled phase II study.

### Objectives

To assess efficacy and safety of prophylactic fibrinogen infusion in CABG patients.

### Study Design

Prospective randomised double-blind placebo-controlled single centre study.

### Inclusion and Exclusion Criteria

CABG patients at Sahlgrenska University Hospital are asked to participate in the study after informed consent. Patients undergoing re-operation, patients with known bleeding disorder, liver disease, or ongoing treatment with drugs influencing the hemostasis (except aspirin) are excluded. Clopidogrel and warfarin are withdrawn at least five days before surgery. Heparin and low-molecular heparin are withdrawn at least 12 hours before surgery.

### Treatment

Fibrinogen (2 g) is infused intravenously to the treatment group during 15 minutes after arrival to the operating room. The control group receives the same amount of placebo infusion.

### Study Plan

CABG patients at Sahlgrenska University Hospital are asked to participate in the study after oral and written information. Patients that have given informed consent are screened regarding plasma concentration of fibrinogen. According to our previous experience approximately 35% of CABG patients have a preoperative fibrinogen plasma concentration of ≦3.7 g/L. Sixty patients with plasma fibrinogen concentration≦3.7 g/L are included in the study and randomized to fibrinogen treatment (2 g) or placebo after arrival to the operating room. The study medication (fibrinogen/placebo) is prepared by the hospital pharmacy. The amount of fibrinogen will increase the plasma concentration with 0.5-0.75 g/L. Accordingly, none of the patients will have a fibrinogen concentration above the upper normal level (4.5 g/L) after treatment. Plasma fibrinogen is measured before infusion, 15 minutes after infusion, during surgery and 15 minutes, 2 hours and 24, 48 and 72 hours after surgery. Conventional screening tests for bleeding and hemostasis (hemoglobin, platelet count, prothrombin time, aPTT, activated clotting time and thromboelastography) are measured at the same time points. Bleeding during the operation and the first 12 postoperative hours is registered. All transfusions of blood products (red blood cells, fresh frozen plasma, and platelets) during hospital stay are registered. All transfusion triggers are predefined. Patients are transfused with red blood cells if blood hemoglobin level decreases to below <80 g/L. Platelets are transfused in patients with on-going bleeding>200 ml/h and platelet count below 75×109 per litre. Plasma are transfused in patients with on-going bleeding>200 ml/h and signs of impaired coagulation on thromboelastometry. All unexpected events during and after the operation are recorded.

### Effect Variables

Primary endpoint is number of transfusions of blood products (packed red cells, plasma, platelets) during hospital stay. Secondary endpoints are plasma levels of fibrinogen and hemostatic variables before, during and after surgery, amount of bleeding the first 12 postoperative hours and cost analysis.

### Event Reports

All events are registered in patient charts and in the CRF. Unexpected events are reported to the research ethics committee.

### Statistics

Based on the findings in the pilot study it is calculated that 60 patients are necessary to show a reduction of transfusions in the treatment group with 50%, with 80% power and a significance level of 0.05.

### Example 6

### Fibrinogen Plasma Concentration and Bleeding Complications after Percutaneous Coronary Interventions

It has recently been demonstrated that bleeding also influences outcome in patients with acute coronary syndromes treated with percutaneous coronary interventions (PCI) (also called Angioplasty, Percutaneous Transluminal Coronary [PTCA], or Balloon Angioplasty). PCI encompasses a variety of procedures used to treat patients with diseased arteries of the heart. One, non-limiting, way of performing PCI may be by threading a slender balloon-tipped tube—a catheter—from an artery in the groin to a trouble spot in an artery of the heart (referred to as percutaneous transluminal coronary angioplasty—also known as PTCA, coronary artery balloon dilation or balloon angioplasty). The balloon is then inflated, compressing the plaque and dilating (widening) the narrowed coronary artery so that blood can flow more easily. This is can be accompanied by inserting an expandable metal stent. Stents are wire mesh tubes used to keep the arteries open after PCI.

It is estimated that approximately 3% of patients undergoing PCI experience major bleeding and up to 20% suffer from less severe bleeding episodes (Feit F, Voeltz M D, Attubato M J, et al. Predictors and impact of major hemorrhage on mortality following percutaneous coronary intervention from the REPLACE-2 Trial. Am J. Cardiol. 2007; 100:1364-9; Eikelboom J W, Mehta S R, Anand S S, Xie C, Fox K A, Yusuf S. Adverse impact of bleeding on prognosis in patients with acute coronary syndromes. Circulation. 2006; 114:774-82). Severe bleeding is an independent predictor for blood transfusion and long-term mortality after PCI. Bleeding in conjunction with PCI is mainly caused by an impaired hemostasis due to the use of anti-coagulant and anti-platelet medication during and after the procedure.

As shown, disclosed in for example 1 and 2, preoperative plasma concentration of fibrinogen is a possible biomarker for bleeding in cardiac surgery patients since it was shown to be an independent predictor of both postoperative bleeding volume and need for transfusion. It is envisaged by the inventors of the present invention that the same relation may exist between plasma concentrations of fibrinogen and bleeding/transfusion requirements after PCI. Thus, it is the inventor's intention to conduct a prospective non-interventional, single—centre study to further elucidate this relation, as described in the following.

### Objective

To assess if preoperative fibrinogen concentration correlate to the incidence of bleeding complications and the need for transfusions after PCI.

### Study Design

Prospective descriptive non-interventional single-centre study.

### Inclusion and Exclusion Criteria

All patients undergoing PCI are asked to participate in the study. There are no pre-set exclusion criteria.

### Treatment

The study is non-interventional.

### Study Plan

800 patients undergoing elective or acute PCI at Sahlgrenska University Hospital are asked to participate in the study after informed consent. Before the procedure the following preoperative variables are recorded: age, gender, body mass index, medication (including anti-coagulation therapy and platelet inhibitors) and blood samples for laboratory analyses (hemoglobin, preoperative plasma fibrinogen concentration, platelet count, aPTT, PT, serum-creatinine) are collected. Peroperatively registered variables includes number type and size of stents, vascular approach (radial or femoral), operation time and perioperative bleeding volume. Bleeding complications will defined according to the international guidelines (Feit F, Voeltz M D, Attubato M J, et al. Predictors and impact of major hemorrhage on mortality following percutaneous coronary intervention from the REPLACE-2 Trial. Am J. Cardiol. 2007; 100:1364-9; Eikelboom J W, Mehta S R, Anand S S, Xie C, Fox K A, Yusuf S. Adverse impact of bleeding on prognosis in patients with acute coronary syndromes. Circulation. 2006; 114:774-82). Transfusions of blood products (red blood cells (RBC), plasma, platelets) during the hospital stay are recorded.

### Statistics

Results will be expressed as mean and standard deviation (SD) or number and percentage (%). Statistical significance is defined as a p-value≦0.05. Fibrinogen levels and other continuous laboratory tests will be compared between patients with and without bleeding complications and between transfused and non-transfused patients with two-sample t-tests. Categorical data will be compared with chi-square tests for categorical data. Independent predictors for transfusion and bleeding complications will be analyzed with multiple logistic regression.

### Example 7

### The Relation Between Preoperative Fibrinogen Plasma Concentration and Thromboelastometry, and Postoperative Bleeding and Transfusion after Coronary Artery Bypass Grafting (CABG)

The data represented in e.g. example 1 and 2 suggest that preoperative plasma concentration of fibrinogen is an independent predictor of bleeding volume and risk of blood transfusion after cardiac surgery. Whole blood thromboelastography assesses global hemostasis. Briefly, in classical thromboelastography, a small blood sample is placed into a cuvette (cup) which is rotated gently to imitate sluggish venous flow and activate coagulation. When a sensor shaft is inserted into the sample a clot forms between the cup and the sensor. The speed and strength of clot formation is measured in various ways, and depends on the activity of the plasmatic coagulation system, platelet function, fibrinolysis and other factors which can be affected by illness, environment and medications. The patterns of changes in strength and elasticity in the clot provide information about how well the blood can perform hemostasis, and how well or poorly different factors are contributing clot formation. Various forms of thromboelastography exists, however, in the present study Rotem® was used, wherein it is the sensor shaft rather than the cup which rotates (Lang T, Bauters A, Braun S L, et al. Multi-centre investigation on reference ranges for ROTEM thromboelastometry. Blood Coagul Fibrinolysis 2005; 16(4):301-10).

The present inventors have investigated the relation between preoperative fibrinogen plasma concentration and thromboelastometry, and postoperative bleeding and transfusion after coronary artery bypass grafting (CABG).

### Material and Methods

Eighty CABG patients (mean age 67±9 years, 75% men) were included in a prospective descriptive study. Preoperatively plasma fibrinogen concentration and thromboelastometry (Rotem®) with Intern, Extern and Fibtem assessment of clotting time (CT), clot formation time (CFT), alpha angle (a) and maximum clot firmness (MCF) were measured. Postoperative bleeding volume and transfusions of packed red blood cells during hospital stay were registered. The correlations between continuous variables were determined with Spearman's rank sum test.

### Results

Mean preoperative fibrinogen plasma concentration was 3.9±0.9 g/L and mean postoperative bleeding was 481±321 ml. Postoperative bleeding volume correlated inversely with preoperative fibrinogen plasma concentration (r=−0.46, p<0.001) and with Fibtem MCF (r=−0.49, p<0.001). Fibrinogen plasma concentration correlated to Fibtem alpha angle (r=0.37, p=0.002) and with Fibtem MCF (r=0.76, p<0.001). 23 patients (28%) received red blood cell transfusion. Transfused patients had lower mean Fibtem MCF (15.1 vs 17.9 mm, p=0.029) and tended to have lower preoperative fibrinogen plasma concentration (3.8 vs 4.2 g/L, p=0.10) than non-transfused patients.

### Conclusions

The results provide further evidence for the key role of fibrinogen concentration for bleeding and blood transfusion requirements after cardiac surgery. Sole measurement of plasma fibrinogen concentration provides similar information as preoperative whole blood thromboelastometry.

### Example 8

### Correlation of Preoperative Fibrinogen Concentration to the Amount of Perioperative Bleedings and Transfusions after Hip Replacement

Excessive bleeding and transfusion of blood products are most common after cardiac surgery and major orthopaedic surgery. It is estimated that approximately 50-60% of patients undergoing hip replacement require blood transfusion. As shown in the previous examples there is an inverse correlation between the preoperative concentration of fibrinogen in plasma and the amount of postoperative bleeding, even in patients above the lower normal limit in cardiac surgery patients. Further, the above-given examples shows that in cardiac surgery patients preoperative plasma concentration of fibrinogen is a possible biomarker for bleeding, since it was an independent predictor of both postoperative bleeding volume and need for transfusion. It is envisaged by the present inventors that such a relation exists with other surgical procedures such as e.g. major orthopaedic surgery. Thus, to further asses if preoperative fibrinogen concentration correlates to the amount of perioperative bleeding and transfusions after hip replacement, the inventors plan to conduct the following clinical study.

### Objective

To assess if preoperative fibrinogen concentration correlate to the amount of perioperative bleeding and transfusions after hip replacement.

### Study Design

Prospective descriptive non-interventional multi-centre study.

### Inclusion and Exclusion Criteria

Hip replacement patients are asked to participate in the study after informed consent. Patients undergoing revision, patients with known bleeding disorder, liver disease, or ongoing treatment with drugs influencing the hemostasis are excluded. Clopidogrel and warfarin are withdrawn at least five days before surgery.

### Treatment

The study is non-interventional.

### Study Plan

200 patients undergoing hip replacement surgery at Sahlgrenska University Hospital, Kungälvs Hospital, Alingsås Hospital and Borås Hospital are asked to participate in the study after oral and written information. The following preoperative variables are recorded: age, gender, body mass index, medication, anticoagulation therapy and laboratory variables (hemoglobin, preoperative plasma fibrinogen concentration, platelet count, aPTT, PT, serum-creatinine). Peroperative variables including surgical approach, type of prosthesis, operation time, perioperative bleeding volume, use of cellsaver and autotranfusion volume are also registered. Postoperative bleeding, defined as wound drainage and amount of transfused red blood cells (RBC), fresh frozen plasma, and platelets during the hospital stay are recorded. RBC transfusions are given when blood haemoglobin level decreases to below <80 g/L. Platelets are transfused in patients with an ongoing bleeding and a platelet count below 75×109 per liter. Plasma is transfused in patients with ongoing bleeding with suspected or confirmed impaired coagulation by thromboelastography.

### Statistics

Results will be expressed as mean and standard deviation (SD) or number and percentage (%). Statistical significance is defined as a p-value≦0.05. Simple linear regression will be used to analyze the relationship between hematological and demographic data and the volume of postoperative bleeding. Multiple linear regression analysis using forward selection will be used to identify factors independently associated with bleeding volume. Comparisons between transfused and non-transfused patients will be done with two-sample t-tests for continuous data and with chi-square tests for categorical data. Independent predictors for transfusion will be analyzed with multiple logistic regression.

### Items

1. A method for preventing perioperative and/or postoperative bleeding in a subject that has not received any therapeutic fibrinogen substitution or fibrinogen replacement therapy 1 week prior to undergoing a surgical procedure, the method comprising administering a substance with fibrinogen-like activity to the subject in an amount that result in a circulating fibrinogen plasma level of from about 1 g/L to about 10 g/L.

2. A method according to item 1, wherein the subject has a preoperative fibrinogen plasma level within the normal range of 2.0 to 4.5 g/L.

3. A method according to item 1 and 2, wherein the amount of the substance with fibrinogen-like activity results in a circulating fibrinogen plasma level of from about 2 g/L to about 9 g/L.

4. A method according to any one of the preceding items, wherein the amount of the substance with fibrinogen-like activity results in a circulating fibrinogen plasma level of from about 2 g/L to about 8 g/L, from about 2.5 g/L to about 7.5 g/L, from about 3 g/L to about 7 g/L, from about 3 g/L to about 6 g/L or from about 3.5 g/L to about 5.5 g/L.

5. A method according to any one of the preceding items, wherein the substance with fibrinogen-like activity is human or recombinant fibrinogen, a derivative or human or recombinant fibrinogen, a chemically modified fibrinogen or a genetically modified fibrinogen.

6. A method according to item 5, wherein the substance with fibrinogen-like activity has 75% or more such as 80% or more, 85% or more, 90% or more, 95% or more or 99% or more identity with human fibrinogen.

7. A method according to any of the preceding items, wherein the subject is a mammal.

8. A method according to item 7, wherein the mammal is a human.

9. A method according to any of the preceding items, wherein the subject has a plasma fibrinogen level of at the most about 6 g/L before any administration of the substance with fibrinogen-like activity.

10. A method according to any one of the preceding items, wherein the subject is expected to undergo a surgical procedure.

11. A method according to item 10, wherein the surgical procedure is one or more of cardiovascular surgery, gynaecological surgery, urological surgery, orthopaedic surgery including hip replacement surgery and back surgery; gastrointestinal surgery, transplantation, tumour surgery, and any other type of surgery with large bleeding risk.

12. A method according to any one of the preceding items, wherein the substance with fibrinogen-like activity is administered in a dose equivalent to from about 0.5 g to about 20 g such as from about 0.5 g to about 15 g, from about 1 g to about 10 g, from about 1 g to about 5 g or from about 1 g to about 2 g of human fibrinogen.

13. A method according to any one of the preceding items, wherein the substance with fibrinogen-like activity is administered in accordance with the following:
i) if the plasma fibrinogen level of the subject is in a range of from about 0.1 g/L-1 g/L then a dose from about 0.30 g/kg body weight to about 0.15 g/kg body weight is administered,
ii) if the plasma fibrinogen level of the subject is in a range of from about 1 g/L-2 g/L then a dose from about 0.25 g/kg body weight to about 0.10 g/kg body weight is administered,
iii) if the plasma fibrinogen level of the subject is in a range of from about 2 g/L-3 g/L then a dose from about 0.20 g/kg body weight to about 0.05 g/kg body weight is administered,
iv) if the plasma fibrinogen level of the subject is in a range of from about 3 g/L-4.5 g/L then a dose from about 0.10 g/kg body weight to about 0.02 g/kg body weight is administered,
v) if the plasma fibrinogen level of the subject is in a range of from about 4.5 g/L-7 g/L then a dose from about 0.10 g/kg body weight to about 0.01 g/kg body weight is administered.

14. A method according to any one of the preceding items, wherein the plasma fibrinogen level after administration is increased with from about 0.1 to about 0.4 g/L per 1 gram of fibrinogen administered.

15. A method according to any one of the preceding items, wherein the plasma fibrinogen level after administration is increased with from about 0.15 to about 0.3 g/L per 1 gram of fibrinogen administered.

16. A method according to any one of the preceding items, wherein the plasma fibrinogen level of the subject is measured at the most 2 weeks before a surgical procedure is carried out.

17. A method according to any one of the preceding items, wherein the plasma fibrinogen level of the subject is measured at the most 1 week such as, e.g. at the most 6 days, at the most 5 days, at the most 4 days, at the most 3 days, at the most 2 days, at the most 1 day, at the most 18 hours, at the most 12 hours, at the most 6 hours, at the most 3 hours, at the most 2 hours, at the most 1 hour, or at the most 30 minutes before a surgical procedure is carried out.

18. A method according to any one of the preceding items, wherein the substance with fibrinogen-like activity is administered at the most 24 hours before the surgical procedure.

19. A method according to any one of the preceding items, wherein the substance with fibrinogen-like activity is administered at the most 18 hours such as, e.g., at the most 12 hours, at the most 9 hours, at the most 6 hours, at the most 3 hours, at the most 1.5 hours or just before the surgical procedure.

20. A method according to any one of the preceding items comprising one or more further administration of the substance with fibrinogen-like activity during the surgical procedure and/or up to 2 days after the surgical procedure.

21. A method according to any one of the preceding items comprising one or more further administration of the substance with fibrinogen-like activity during the surgical procedure and/or up to 1 day such as, e.g., up to 18 hours, up to 12 hours, up to 6 hours, up to 3 hours, up to 1.5 hours or up to 1 hour after the surgical procedure.

22. A method according to any one of the preceding items comprising administration of a further therapeutically or prophylactically active substance.

23. A method according to item 22, wherein the active substance is a haemostatic agent such as aprotinin or tranexamic acid.

24. A method according to item 22, wherein the active substance is a platelet inhibitor such as clopidogrel, acetylsalicylic acid, vasopressin, heparin, low molecular weight heparin, warfarin, a GP IIb/IIIa modulator, thrombin inhibitor, fibrinogen inducing substance including IL2b.

25. A method according to any of the preceding items, wherein any treatment with low molecular weight heparin is discontinued at least 12 hours before subjecting the subject to surgery.

26. A method according to any of the preceding items, wherein any treatment with clopidogrel and/or warfarin are discontinued at least 5 days before subjecting the subject to surgery.

27. A method according to any one of the preceding items, wherein the substance with fibrinogen-like activity is administered in the form of a pharmaceutical composition.

28. A method according to item 27, wherein the pharmaceutical composition is for parenteral administration.

29. A method according to item 27 or 28, wherein the pharmaceutical composition comprises the substance with fibrinogen-like activity and an aqueous medium.

30. A method according to item 27, wherein the substance with fibrinogen-like activity is dispersed in the aqueous medium substantially immediately before administration.

31. A method according to any one of items 27-30, wherein the composition comprises a pH adjusting agent, a stabilizing agent, a solubilizing agent, and/or a osmotic pressure adjusting agent.

32. A method according to any one of items 27-31, wherein the composition comprises a pharmaceutically acceptable salt.

33. A method according to item 32, wherein the salt is a phosphate, a carbonate, an amino acid salt including a lysinate or a glycinate, a salt of a carboxylic acid including an acetate, a butyrate, a valerate, a succinate, a hemisuccinate; a cyprionate or a trometamole salt or a combination thereof.

34. Use of a substance with fibrinogen-like activity for the manufacture of a pharmaceutical composition for the prevention of perioperative and/or postoperative bleeding in a subject that has not received any therapeutic fibrinogen substitution or fibrinogen replacement therapy 1 week prior to the perioperative and/or postoperative bleeding.

35. Use according to item 34, wherein the subject has a preoperative fibrinogen plasma level within the normal range of 2.0 to 4.5 g/L.

36. Use according to items 34 and 35, wherein the substance with fibrinogen-like activity is human or recombinant fibrinogen, a derivative or human or recombinant fibrinogen, a chemically modified fibrinogen or a genetically modified fibrinogen.

37. Use according to items 34-36, wherein the substance with fibrinogen-like activity has 75% or more such as 80% or more, 85% or more, 90% or more, 95% or more or 99% or more identity with human fibrinogen (SEQ ID No: 1).

38. Use according to any of items 34-37, wherein the subject is a mammal.

39. Use according to item 38, wherein the mammal is a human.

40. Use according to any of items 34-39, wherein the subject has a plasma fibrinogen level of at the most about 6 g/L before any administration of the substance with fibrinogen-like activity.

41. Use according to any one of items 34-40, wherein the subject is expected to undergo a surgical procedure.

42. Use according to item 41, wherein the surgical procedure is one or more of cardiovascular surgery, gynaecological surgery, urological surgery, orthopaedic surgery including hip replacement surgery and back surgery; gastrointestinal surgery, transplantation, tumour surgery, and any other type of surgery with large bleeding risk.

43. Use according to any one of items 37-42, wherein the substance with fibrinogen-like activity is administered in a dose equivalent to from about 0.5 g to about 20 g such as from about 0.5 g to about 15 g, from about 1 g to about 10 g, from about 1 g to about 5 g or from about 1 g to about 2 g of human fibrinogen.

44. Use according to any one of items 34-43, wherein the substance with fibrinogen-like activity is administered in accordance with the following:
i) if the plasma fibrinogen level of the subject is in a range of from about 0.1 g/L-1 g/L then a dose from about 0.30 g/kg body weight to about 0.15 g/kg body weight is administered,
ii) if the plasma fibrinogen level of the subject is in a range of from about 1 g/L-2 g/L then a dose from about 0.25 g/kg body weight to about 0.10 g/kg body weight is administered,
iii) if the plasma fibrinogen level of the subject is in a range of from about 2 g/L-3 g/L then a dose from about 0.20 g/kg body weight to about 0.05 g/kg body weight is administered,
iv) if the plasma fibrinogen level of the subject is in a range of from about 3 g/L-4.5 g/L then a dose from about 0.10 g/kg body weight to about 0.02 g/kg body weight is administered,
v) if the plasma fibrinogen level of the subject is in a range of from about 4.5 g/L-7 g/L then a dose from about 0.10 g/kg body weight to about 0.01 g/kg body weight is administered.

45. Use according to any one of items 34-44, wherein the plasma fibrinogen level after administration is increased with from about 0.1 to about 0.4 g/L per 1 gram of fibrinogen administered.

46. Use according to any one of items 34-45, wherein the plasma fibrinogen level after administration is increased with from about 0.15 to about 0.3 g/L per 1 gram of fibrinogen administered.

47. Use according to any one of items 34-46, wherein the plasma fibrinogen level of the subject is measured at the most 2 weeks before a surgical procedure is carried out.

48. Use according to any one of items 34-47, wherein the plasma fibrinogen level of the subject is measured at the most 1 week such as, e.g. at the most 6 days, at the most 5 days, at the most 4 days, at the most 3 days, at the most 2 days, at the most 1 day, at the most 8 hours, at the most 12 hours, at the most 6 hours, at the most 3 hours, at the most 2 hours, at the most 1 hour, or at the most 30 minutes before a surgical procedure is carried out.

49. Use according to any one of items 34-48, wherein the substance with fibrinogen-like activity is administered at the most 24 hours before the surgical procedure.

50. Use according to any one of items 34-49, wherein the substance with fibrinogen-like activity is administered at the most 18 hours such as, e.g., at the most 12 hours, at the most 9 hours, at the most 6 hours, at the most 3 hours, at the most 1.5 hours or just before the surgical procedure.

51. Use according to any one of items 34-50 comprising one or more further administration of the substance with fibrinogen-like activity during the surgical procedure and/or up to 2 days after the surgical procedure.

52. Use according to any one of items 34-51 comprising one or more further administration of the substance with fibrinogen-like activity during the surgical procedure and/or up to 1 day such as, e.g., up to 18 hours, up to 12 hours, up to 6 hours, up to 3 hours, up to 1.5 hours or up to 1 hour after the surgical procedure.

53. Use according to any one of items 34-52 comprising administration of a further therapeutically or prophylactically active substance.

54. Use according to item 53, wherein the active substance is a haemostatic agent such as aprotinin or transexamic acid.

55. Use according to item 53, wherein the active substance is a platelet inhibitor such as clopidogrel, acetylsalicylic acid, vasopressin, heparin, low molecular weight heparin, warfarin, a GP IIb/IIIa modulator, thrombin inhibitor, fibrinogen inducing substance including IL2b.

56. Use according to any one of items 34-55, wherein any treatment with low molecular weight heparin is discontinued at least 12 hours before subjecting the subject to surgery.

57. Use according to any one of items 34-56, wherein any treatment with clopidogrel and/or warfarin are discontinued at least 5 days before subjecting the subject to surgery.

58. Use according to any one of items 34-57, wherein the substance with fibrinogen-like activity is administered in the form of a pharmaceutical composition.

59. Use according to item 58, wherein the pharmaceutical composition is for parenteral administration.

60. Use according to item 58 or 59, wherein the pharmaceutical composition comprises the substance with fibrinogen-like activity and an aqueous medium.

61. Use according to item 60, wherein the substance with fibrinogen-like activity is dispersed in the aqueous medium substantially immediately before administration.

62. Use according to any one of items 34-61, wherein the composition comprises a pH adjusting agent, a stabilizing agent, a solubilizing agent, and/or a osmotic pressure adjusting agent.

63. Use according to any one of items 34-62, wherein the composition comprises a pharmaceutically acceptable salt.

64. Use according to item 63, wherein the salt is a phosphate, a carbonate, an amino acid salt including a lysinate or a glycinate, a salt of a carboxylic acid including an acetate, a butyrate, a valerate, a succinate, a hemisuccinate; a cyprionate or a trometamole salt or a combination thereof.

65. A kit comprising a substance with fibrinogen-like activity, an aqueous medium and instructions for using the kit in a method as defined in any one of items 1-33.

66. A method for prevention of perioperative and/or postoperative bleeding, the method comprising

i) sampling a biological sample from a subject,
ii) measuring fibrinogen content in the sample, and
iii) comparing the value obtained in step ii) with the normal level.

67. A method for evaluating the risk of perioperative and/or postoperative bleeding, the method comprising

i) sampling a biological sample from a subject,
ii) measuring fibrinogen content in the sample, and
iii) identifying the risk by use of a plot of plasma fibrinogen level versus risk of blood transfusion.

68. A method for evaluating the need for blood or plasma transfusion to a subject expected to undergo a surgical procedure, the method comprising

i) sampling a biological sample from a subject,
ii) measuring fibrinogen content in the sample, and
iii) comparing the value obtained in step ii) with the normal level.

69. A method according to items 66-68, wherein the subject has not received any therapeutic fibrinogen substitution or fibrinogen replacement therapy 1 week prior to the evaluation or prevention defined in any one of items 66-68.

70. A method according to item 69, wherein the subject has a preoperative fibrinogen plasma level within the normal range of 2.0 to 4.5 g/L.

71. A method according to any one of items 66-70, wherein the biological sample is a blood or plasma sample.

72. A method according to any one of items 66-71, wherein the subject is a human.

73. A method according to any one of items 66-72, wherein the subject is expected to undergo a surgical procedure.

74. A method according to any one of items 66-73, wherein the surgical procedure is one or more of cardiovascular surgery, gynaecological surgery, urological surgery, orthopaedic surgery including hip replacement surgery and back surgery; gastrointestinal surgery, transplantation, tumour surgery and surgery with large bleeding risk.

75. A method according to any one of items 66-74 for diagnosing the need for fibrinogen therapy using the measured value obtained in step ii) as an indicator.

76. Use of plasma fibrinogen level as a biomarker for the prevention of perioperative and/or postoperative bleeding in a subject undergoing a surgical procedure.

77. Use of plasma fibrinogen level to predict the risk of perioperative and/or postoperative bleeding in a subject undergoing a surgical procedure.

78. Use of plasma fibrinogen level to predict the need for plasma or blood transfusion in a subject undergoing a surgical procedure.

79. Use according to items 76-78, wherein the subject has not received any therapeutic fibrinogen substitution or fibrinogen replacement therapy 1 week prior to the use as defined in any one of items 66-68.

80. Use according to item 79, wherein the subject has a preoperative fibrinogen plasma level within the normal range of 2.0 to 4.5 g/L.

1-60. (canceled)

61. A method for preventing perioperative and/or postoperative bleeding associated with a surgical procedure in a subject having a plasma fibrinogen level equal to or above a lower normal limit of 2 g/L, the method comprising administering fibrinogen to the subject in an amount that results in a circulating fibrinogen plasma level of from about 2 g/L to about 10 g/L.

62. The method of claim 61, wherein administering fibrinogen to the subject comprises administering fibrinogen to the subject in an amount that results in a circulating fibrinogen plasma level selected from above about 2.5 g/L, above about 3 g/L, above about 3.5 g/L, above about 4 g/L, above about 4 g/L, above about 6 g/L, above about 7 g/L, above about 8 g/L, and above about 9 g/L.

63. The method of claim 61, wherein, prior to administering fibrinogen for preventing perioperative and/or postoperative bleeding, the subject has not previously received a fibrinogen treatment to increase the subject's plasma fibrinogen level to within a normal range.

64. The method of claim 61, further characterized in that the subject is not suffering from a congenital or acquired fibrinogen deficiency.

65. The method of claim 61, further characterized in that the subject has not received any fibrinogen treatment 1 week or more prior to the surgical procedure.

66. The method of claim 61, further characterized in that the subject has a preoperative fibrinogen plasma level within a normal range of from about 2 g/L to about 4.5 g/L.

67. The method of claim 61, wherein administering fibrinogen to the subject comprises administering fibrinogen to the subject in an amount that results in a circulating fibrinogen plasma level of from about 2 g/L to about 9 g/L.

68. The method of claim 61, wherein administering fibrinogen to the subject comprises administering fibrinogen to the subject in an amount that results in a circulating fibrinogen plasma level selected from about 2 g/L to about 8 g/L, about 2.5 g/L to about 7.5 g/L, about 3 g/L to about 7 g/L, about 3 g/L to about 6 g/L, about 3.5 g/L to about 5.5 g/L.

69. The method of claim 61, wherein administering fibrinogen to the subject comprises administering one or more of human fibrinogen, recombinant human fibrinogen, a derivative of human fibrinogen, a derivative of recombinant human fibrinogen, a chemically modified fibrinogen, and a genetically modified fibrinogen.

70. The method of claim 69, further characterized in that the fibrinogen is selected from a molecule that exhibits a homology selected from 75% or more, 80% or more, 85% or more, 90% or more, 95% or more, and 99% or more to human fibrinogen.

71. The method of claim 61, wherein the subject is a mammal.

72. The method of claim 61, wherein the subject is human.

73. The method of claim 61, further characterized in that the subject exhibits a fibrinogen level of at most 6 g/L prior to administration of fibrinogen for preventing perioperative and/or postoperative bleeding.

74. The method of claim 61, further characterized in that the surgical procedure is selected from one or more of cardiovascular surgery, gynecological surgery, urological surgery, orthopedic surgery, including hip replacement surgery and back surgery, gastrointestinal surgery, transplantation, tumor surgery, open surgical procedures and minimally invasive interventions, including laparoscopic, thoracoscopic, endoscopic surgical interventions, and catheter-based interventions.

75. The method of claim 61, further characterized in that the surgical procedure is selected from cardiovascular surgery and the cardiovascular surgery is further selected from one or more of open heart surgery with or without cardiopulmonary bypass, coronary artery bypass surgery, off-pump coronary artery bypass surgery, minimally invasive direct coronary artery bypass surgery, valve surgery, aortic surgery, and combinations thereof.

76. The method of claim 61, further characterized in that the surgical procedure is selected from an ophthalmic surgical procedure and a neurosurgical procedure.

77. The method of claim 61, wherein administering fibrinogen to the subject comprises administering fibrinogen to the subject at a dose equivalent selected from about 0.5 g to about 20 g, from about 0.5 g to about 15 g, from about 1 g to about 10 g, from about 1 g to about 5 g, and from about 1 g to about 2 g of human fibrinogen.

78. The method of claim 61, wherein administering fibrinogen to the subject comprise administering fibrinogen according to a dosing regimen selected from;
(i) administering a dose of fibrinogen to the subject of from about 0.20 g/kg body weight to about 0.05/g/kg body weight where the plasma fibrinogen level of the subject prior to the surgical procedure is in the range of from about 2 g/L to about 3 g/L;
(ii) administering a dose of fibrinogen to the subject of from about 0.10 g/kg body weight to about 0.02/g/kg body weight where the plasma fibrinogen level of the subject prior to the surgical procedure is in the range of from about 3 g/L to about 4.5 g/L; and
(iii) administering a dose of fibrinogen to the subject of from about 0.10 g/kg body weight to about 0.01/g/kg body weight where the plasma fibrinogen level of the subject prior to the surgical procedure is in the range of from about 4.5 g/L to about 7 g/L.

79. The method of claim 61, wherein administering fibrinogen to the subject comprises administering fibrinogen to the subject such that the plasma fibrinogen level in the subject increases from about 0.1 g/L to about 0.4 g/L per 1 gram fibrinogen administered.

80. The method of claim 61, wherein administering fibrinogen to the subject comprises administering fibrinogen to the subject such that the plasma fibrinogen level in the subject increases from about 0.15 g/L to about 0.3 g/L per 1 gram fibrinogen administered.

81. The method of claim 61, further comprising measuring the plasma fibrinogen level of the subject at most 2 weeks prior to the surgical procedure.

82. The method of claim 81, wherein measuring the plasma fibrinogen level of the subject takes place at a time selected from at most 1 week, at most 6 days, at most days, at most 4 days, at most 3 days, at most 2 days, at most 1 day, at most 18 hours, at most 12 hours, at most 6 hours, at most 3 hours, at most 2 hours, at most 1 hour, and at most 30 minutes prior to the surgical procedure.

83. The method of claim 61, wherein administering fibrinogen to the subject comprises administering fibrinogen to the subject at most 240 hours prior to the surgical procedure.

84. The method of claim 61, wherein administering fibrinogen to the subject comprises administering fibrinogen to the subject at most 24 hours prior to the surgical procedure.

85. The method of claim 61, wherein administering fibrinogen to the subject comprises administering fibrinogen to the subject at a timing selected from at most 18 hours, at most 12 hours, at most 9 hours, at most 6 hours, at most 3 hours, and at most 1.5 hours prior to the surgical procedure.

86. The method of claim 61, further comprising administering an additional dose of fibrinogen to the subject at a timing selected from during the surgical procedure, up to 2 days after the surgical procedure, and both during the surgical procedure and up to 2 days after the surgical procedure.

87. The method of claim 61, further comprising administering an additional dose of fibrinogen to the subject at a timing selected from during the surgical procedure, up to 1 day after the surgical procedure, and both during the surgical procedure and up to 1 day after the surgical procedure, wherein up to 1 day after the surgical procedure is selected from up to 24 hours, up to 18 hours, up to 12 hours, up to 6 hours, up to 3 hours, up to 1.5 hours, and up to 1 hour after the surgical procedure.

88. The method of claim 61, further comprising administering an additional therapeutically or prophylactically active substance to the subject.

89. The method of claim 88, wherein administering an additional therapeutically or prophylactically active substance to the subject comprises administering a haemostatic agent.

90. The method of claim 88, wherein administering an additional therapeutically or prophylactically active substance to the subject comprises administering a substance selected from clopidogrel, acetylsalicylic acid, vasopressin, heparin, low molecular weight heparin, warfarin, a GP IIb/IIIa modulator, a thrombin inhibitor, a fibrinogen inducing substance, and IL2b.

91. The method of claim 61, wherein administering fibrinogen to the subject comprises administering a pharmaceutical composition comprising fibrinogen to the subject.

92. The method of claim 91, further characterized in that the pharmaceutical composition is a composition suitable for parenteral administration.

93. The method of claim 91, further characterized in that the pharmaceutical composition comprises fibrinogen in an aqueous medium.

94. The method of claim 93, further characterized in that the pharmaceutical composition is formed by dispersing the fibrinogen in the aqueous medium substantially immediately before administration.

95. The method of claim 91, further characterized in that the pharmaceutical composition comprises one or more constituents selected from a pH adjusting agent, a stabilizing agent, a solubilizing agent, an osmotic pressure adjusting agent, and a pharmaceutically acceptable salt.

96. The method of claim 95, further characterized in that the pharmaceutical composition comprises one or more pharmaceutically acceptable salts and said one or more pharmaceutically acceptable salts are selected from a phosphate, a carbonate, an amino acid salt, lysinate, glysinate, a carboxylic acid salt, an acetate, a butyrate, a valerate, a succinate, a hemisuccinate, a cyprionate, and a trometamole salt.

97. A method for manufacturing a medication for inhibiting perioperative and/or postoperative bleeding associated with a surgical procedure, the method comprising preparing a pharmaceutical composition comprising fibrinogen.

98. The method of claim 97, further characterized in that the surgical procedure is selected from one or more of cardiovascular surgery, gynecological surgery, urological surgery, orthopedic surgery, including hip replacement surgery and back surgery, gastrointestinal surgery, transplantation, tumor surgery, open surgical procedures and minimally invasive interventions, including laparoscopic, thoracoscopic, endoscopic surgical interventions, and catheter-based interventions.

99. The method of claim 97, further characterized in that the surgical procedure is selected from cardiovascular surgery and the cardiovascular surgery is further selected from one or more of open heart surgery with or without cardiopulmonary bypass, coronary artery bypass surgery, off-pump coronary artery bypass surgery, minimally invasive direct coronary artery bypass surgery, valve surgery, aortic surgery, and combinations thereof.

100. The method of claim 97, further characterized in that the surgical procedure is selected from an ophthalmic surgical procedure and a neurosurgical procedure.

101. A kit comprising:
a pharmaceutical composition suitable for administration to a subject, the pharmaceutical composition comprising fibrinogen; and
instructions for administering the pharmaceutical composition to the subject for inhibiting perioperative and/or postoperative bleeding associated with a surgical procedure.

102. The kit according to claim 101, wherein the fibrinogen is selected from one or more of human fibrinogen, recombinant human fibrinogen, a derivative of human fibrinogen or recombinant human fibrinogen, a chemically modified fibrinogen, and a genetically modified fibrinogen.

103. The kit according to claim 102, wherein the fibrinogen is selected from a molecule that exhibits a homology selected from 75% or more, 80% or more, 85% or more, 90% or more, 95% or more, and 99% or more to human fibrinogen.

104. The kit of claim 101, wherein the kit comprises a fibrinogen, an aqueous medium in which the fibrinogen can be dispersed to form the pharmaceutical composition that may be administered to a subject, and instructions for preparing the pharmaceutical composition for administration to the subject.

105. The kit of claim 104, wherein the fibrinogen is provided in dry or lyophilized from.

106. The kit of claim 101, wherein the pharmaceutical formulation is provided as a solution.

107. The kit of claim 101, wherein the pharmaceutical formulation further comprises one or more stabilizing agents.

108. The kit of claim 101, further comprising a device for parenteral administration of the pharmaceutical composition.

109. The kit of claim 108, wherein the device is suitable for intravenous administration of the pharmaceutical composition.

110. A method for determining the risk of perioperative and/or postoperative bleeding in a subject that will undergo a surgical procedure, the method comprising:
(i) taking a biological sample from the subject;
(ii) measuring fibrinogen content of the biological sample; and
(iii) comparing the value obtained in step (ii) with a normal level.

111. A method for determining the risk of perioperative and/or postoperative bleeding in a subject that will undergo a surgical procedure, the method comprising:
(i) taking a biological sample from the subject;
(ii) measuring fibrinogen content of the biological sample;
(iii) providing a plot of plasma fibrinogen content versus risk of perioperative and/or postoperative bleeding; and
(iv) determining where the fibrinogen content measured in step (ii) falls on the plot provided in step (iii); and
(v) identifying the risk of blood transfusion based on where the value obtained in step (ii) falls on the plot.

112. A method for evaluating the potential need for a blood transfusion in a subject that will undergo a surgical procedure, the method comprising:
(i) taking a biological sample from the subject;
(ii) measuring fibrinogen content of the biological sample; and
(iii) comparing the value obtained in step (ii) with a normal level.

113. The method of claim 110, further characterized in that the subject exhibits a plasma fibrinogen level equal to or above a lower normal level for plasma fibrinogen prior to taking of the biological sample.

114. The method of claim 111, further characterized in that the subject exhibits a plasma fibrinogen level equal to or above a lower normal level for plasma fibrinogen prior to taking of the biological sample.

115. The method of claim 112, further characterized in that the subject exhibits a plasma fibrinogen level equal to or above a lower normal level for plasma fibrinogen prior to taking of the biological sample.
